(19) United States
(12) Patent Application Publication
(10) Pub. No.: US 2026/0294945 A1
(43) Pub. Date: Oct. 1, 2026

(54) COMPOSITIONS COMPRISING HUMAN MILK OLIGOSACCHARIDES FOR USE IN A SUBJECT TO SUPPORT BRAIN DEVELOPMENT AND/OR COGNITIVE DEVELOPMENT

(71) Applicant: SOCIÉTÉ DES PRODUITS NESTLÉ S.A., Vevey (CH)

(72) Inventors: Tinu Mary SAMUEL, La Tour De Peilz (CH); Norbert SPRENGER, Savigny (CH); Jonas HAUSER, Lausanne (CH); Weili LIN, Chapel Hill, NC (US); Seoyoon CHO, Morrisville, NC (US); Ziliang ZHU, Chapel Hill, NC (US); Weiyan YIN, Chapel Hill, NC (US); Tengfei LI, Chapel Hill, NC (US); Hongtu ZHU, Chapel Hill, NC (US); Hanne Lore Paula TYTGAT, Zurich (CH); Purva RAJHANS, Lausanne (CH)

(21) Appl. No.: 19/478,633

(22) PCT Filed: Apr. 26, 2024
(86) PCT No.: PCT/EP2024/061691

§ 371 Date: Oct. 24, 2025

Related U.S. Application Data
(60) Provisional application No. 63/462,613, filed on Apr. 28, 2023.

(51) Int. Cl. A61K 31/702; A61K 35/745; A61P 25/00

(52) U.S. Cl. CPC A61K 31/702; A61K 35/745; A61P 25/00

(57) ABSTRACT

The invention provides a nutritional composition comprising at least one lacto-N-fucopentaose, especially lacto-N-fucopentaose-I (LNFP-I) or a combination of lacto-N-fucopentaose-I (LNFP-I), lacto-N-fucopentaose-II (LNFP-II) and lacto-N-fucopentaose-III (LNFP-III), for use in improving brain development and/or cognitive development in a subject. A nutritional composition comprising at least one lacto-N-fucopentaose, especially lacto-N-fucopentaose-I (LNFP-I) or a combination of lacto-N-fucopentaose-I (LNFP-I), lacto-N-fucopentaose-II (LNFP-II) and lacto-N-fucopentaose-III (LNFP-III), and Bifidobacterium kashiwanohense is also provided.

### FIELD OF THE INVENTION

This invention relates to nutritional compositions comprising at least one human milk oligosaccharide for use in a subject to support brain development and/or cognitive development. In particular, the nutritional composition comprises at least one lacto-N-fucopentaose (LNFP) such as lacto-N-fucopentaose-I (LNFP-I). The brain development and/or cognitive development comprises the development of auditory brain networks, sensorimotor brain networks, default mode brain networks and language.

### BACKGROUND OF THE INVENTION

Identifying the potential relationship between human milk oligosaccharides (HMOs) and early brain development has gained substantial interest in recent years. Preliminary evidence based on animal studies have indicated that HMOs could play a significant role in the central nervous system.

The potential interactions between HMOs and gut microbiota have been implicated since HMOs are largely metabolized in the intestinal tract. Gut microbiota composition and particular HMOs (e.g. 2′-FL) have shown to be independently associated with infant cognition. For example, using chronic oral administration of 2′-FL to rodents, Vazquez et al. demonstrated that the treated animals exhibit significantly better learning and working memory function (Vazquez et al., 2015, Journal of nutritional biochemistry, 26(5):455-465). However, there is a paucity of data on the potential effects of other HMOs as well as the interactions between gut microbiota and HMOs on brain functional development in infancy and early childhood. To the best of our knowledge, the present study is the first study to determine the association of the HMOs LNFP, especially lacto-N-fucopentaose-I (LNFP-I) on cognitive development and to determine how LNFP, especially LNFP-I and Bifidobacterium kashiwanohense interact with each other and their associations with cognitive development in infants.

WO 2014/100022 relates to a nutritional composition for use in enhancing learning and memory in an individual, wherein composition comprises at least one HMO. This work shows an example of gamma amino butyric acid (GABA) production by babies' fecal microbiota in the presence of LNnT. It however fails to show any evidence of the effect of this neurotransmitter on the brain. This is further difficult to expect, considering that GABA has been proposed to not cross the blood brain barrier (Van Gelder and Elliott, 1958 Neurochem. December; 3(2): 139-43; Kuriyama and Sze, (1971) Neuropharmacology January; 10(I): 103-8; Knudsen et al., (1988) Hepatol. 1988 April; 6 (2): 187-92). Finally, the example supports a role for LNnT, which is a neutral HMO, but not of any other HMOs, and while some other HMOs are sharing similarities with LNnT, it is also true that many are dissimilar in either their building block or their structure, rendering the extension of the findings obtained with LNnT to other HMOs difficult to support.

There is a need for further compositions which can deliver such health benefits. There is a need to deliver such health benefits in the subject in a manner that does not induce side effects and/or in a manner that is easy to deliver, and well accepted by the parents or health care practitioners. There is also a need to deliver such benefits in a manner that keeps the cost of such delivery reasonable and affordable by most.

### SUMMARY OF THE INVENTION

The present inventors have surprisingly found that the HMO lacto-N-fucopentaose (LNFP), such as lacto-N-fucopentaose-I (LNFP-I) are significantly associated with brain and cognitive development, and in particular, the development of auditory brain networks, sensorimotor brain networks, default mode brain networks and language, in infants and children. Furthermore, the inventors have surprisingly found that the interactions between at least one LNFP (especially LNFP-I) and Bifidobacterium kashiwanohense is significantly associated with brain and cognitive development, and in particular, the development of expressive language, in infants and children.

Accordingly, in a first aspect, the invention provides a nutritional composition comprising at least one LNFP, especially lacto-N-fucopentaose-I (LNFP-I), for use in improving brain development and/or cognitive development in a subject.

In a further aspect, the invention provides at least one LNFP, especially lacto-N-fucopentaose-I (LNFP-I), for use in improving brain development and/or cognitive development in a subject.

In a further aspect, the invention provides the use of a nutritional composition comprising at least one LNFP, especially lacto-N-fucopentaose-I (LNFP-I), for improving brain development and/or cognitive development in a subject.

In a further aspect, the invention provides the use of at least one LNFP, especially lacto-N-fucopentaose-I (LNFP-I), for improving brain development and/or cognitive development in a subject.

In a further aspect, the invention provides a method of improving brain development and/or cognitive development in a subject, the method comprising administering a nutritional composition comprising at least one LNFP, especially lacto-N-fucopentaose-I (LNFP-I), to the subject.

In a further aspect, the invention provides a method of improving brain development and/or cognitive development in a subject, the method comprising administering at least one LNFP, especially lacto-N-fucopentaose-I (LNFP-I), to the subject.

In some embodiments, the at least one LNFP is lacto-N-fucopentaose-I (LNFP-I), lacto-N-fucopentaose-II (LNFP-II), lacto-N-fucopentaose-III (LNFP-III), lacto-N-fucopentaose-V (LNFP-V) or any combination thereof. Suitably, the at least one LNFP is LNFP-I. Suitably, the at least one LNFP is a combination of LNFP-I and LNFP-II. Suitably, the at least one LNFP is a combination of LNFP-I and LNFP-III. Suitably, the at least one LNFP is a combination of LNFP-I, LNFP-II and LNFP-III.

Accordingly, in a further aspect, the invention provides a nutritional composition comprising a combination of lacto-N-fucopentaose-I (LNFP-I), lacto-N-fucopentaose-II (LNFP-II) and lacto-N-fucopentaose-III (LNFP-III), for use in improving brain development and/or cognitive development in a subject.

In a further aspect, the invention provides a combination of lacto-N-fucopentaose-I (LNFP-I), lacto-N-fucopentaose-II (LNFP-II) and lacto-N-fucopentaose-III (LNFP-III), for use in improving brain development and/or cognitive development in a subject.

In a further aspect, the invention provides the use of a nutritional composition comprising a combination of lacto-N-fucopentaose-I (LNFP-I), lacto-N-fucopentaose-II (LNFP-II) and lacto-N-fucopentaose-III (LNFP-III), for improving brain development and/or cognitive development in a subject.

In a further aspect, the invention provides the use of a combination of lacto-N-fucopentaose-I (LNFP-I), lacto-N-fucopentaose-II (LNFP-II) and lacto-N-fucopentaose-III (LNFP-III) for improving brain development and/or cognitive development in a subject.

In a further aspect, the invention provides a method of improving brain development and/or cognitive development in a subject, the method comprising administering a nutritional composition comprising a combination of lacto-N-fucopentaose-I (LNFP-I), lacto-N-fucopentaose-II (LNFP-II) and lacto-N-fucopentaose-III (LNFP-III), to the subject.

In a further aspect, the invention provides a method of improving brain development and/or cognitive development in a subject, the method comprising administering a combination of lacto-N-fucopentaose-I (LNFP-I), lacto-N-fucopentaose-II (LNFP-II) and lacto-N-fucopentaose-III (LNFP-III) to the subject.

In some embodiments, the improvement in brain development and/or cognitive development comprises or consists of an improvement in the development of auditory brain networks, sensorimotor brain networks, default mode brain networks, language, fine motor skills, or combinations thereof.

In some embodiments, the invention provides a nutritional composition comprising lacto-N-fucopentaose-I (LNFP-I) for use in improving brain development and/or cognitive development in a subject.

In some embodiments, the invention provides lacto-N-fucopentaose-I (LNFP-I) for use in improving brain development and/or cognitive development in a subject.

In some embodiments, the invention provides the use of a nutritional composition comprising lacto-N-fucopentaose-I (LNFP-I) for improving brain development and/or cognitive development in a subject.

In some embodiments, the invention provides the use of lacto-N-fucopentaose-I (LNFP-I) for improving brain development and/or cognitive development in a subject.

In some embodiments, the invention provides the use of a combination of lacto-N-fucopentaose-I (LNFP-I), lacto-N-fucopentaose-II (LNFP-II) and lacto-N-fucopentaose-III (LNFP-III) for improving brain development and/or cognitive development in a subject, wherein the improvement in brain development and/or cognitive development comprises or consists of an improvement in the development of fine motor skills.

In some embodiments, the improvement in language development comprises or consists of an improvement in receptive language and/or expressive language based on a score of Mullen sub-scale or of the Bayley sub-scale.

In some embodiments, the improvement in language development comprises or consists of an improvement in receptive language and/or expressive language based on a score of Mullen sub-scale.

In some embodiments, the improvement in language development comprises or consists of an improvement in expressive language based on a score of Mullen sub-scale or of the Bayley sub-scale.

In some embodiments, the improvement in brain development and/or cognitive development comprises or consists of an improvement in expressive language based on a score of Mullen sub-scale or of the Bayley sub-scale. Suitably, the nutritional composition is for use in improving brain development and/or cognitive development in a subject by increasing the abundance of Bifidobacterium kashiwanohense in the gastrointestinal tract of the subject. In a preferred embodiment, the subject is born of an A-tetrasaccharide negative mother and/or the subject is fed with a mother's milk lacking A-tetrasaccharide.

In some embodiments, the nutritional composition further comprises B. kashiwanohense. Suitably, the B. kashiwanohense is present in an amount of from 10e3 to 10e12 cfu.

In some embodiments, the at least one LNFP is present in a total amount of from 25 mg/L to 5000 mg/L of the nutritional composition or of from 0.02-3.75 g/100 g of the nutritional composition.

In some embodiments, LNFP-I is present in a total amount of from 25 mg/L to 5000 mg/L of the nutritional composition or of from 0.02-3.75 g/100 g of the nutritional composition.

In some embodiments, LNFP-I, LNFP-I and LNFP-III are present in a total amount of from 25 mg/L to 5000 mg/L of the nutritional composition or of from 0.02-3.75 g/100 g of the nutritional composition.

In some embodiments, the subject is an infant, a young child or a child.

In some embodiments, said nutritional composition is an infant formula, a starter infant formula, a follow-on or follow-up infant formula, a baby food, an infant cereal composition, a growing-up milk, a fortifier or a supplement.

In a further aspect, the invention provides the use of a nutritional composition comprising at least one LNFP, especially lacto-N-fucopentaose-I (LNFP-I), for increasing the abundance of B. kashiwanohense in the gastrointestinal tract of a subject.

In some embodiments, the invention provides the use of a nutritional composition comprising lacto-N-fucopentaose-I (LNFP-I) for increasing the abundance of B. kashiwanohense in the gastrointestinal tract of a subject.

In a further aspect, the invention provides the use of at least one LNFP, especially lacto-N-fucopentaose-I (LNFP-I), for increasing the abundance of B. kashiwanohense in the gastrointestinal tract of a subject.

In some embodiments, the invention provides the use of lacto-N-fucopentaose-I (LNFP-I) for increasing the abundance of B. kashiwanohense in the gastrointestinal tract of a subject.

In a further aspect, the invention provides the use of a nutritional composition comprising a combination of lacto-N-fucopentaose-I (LNFP-I), lacto-N-fucopentaose-II (LNFP-II) and lacto-N-fucopentaose-III (LNFP-III), for increasing the abundance of B. kashiwanohense in the gastrointestinal tract of a subject.

In a further aspect, the invention provides the use of a combination of lacto-N-fucopentaose-I (LNFP-I), lacto-N-fucopentaose-II (LNFP-II) and lacto-N-fucopentaose-III (LNFP-III), for increasing the abundance of B. kashiwanohense in the gastrointestinal tract of a subject.

In some embodiments, the subject is:
a) born of an A-tetrasaccharide negative mother; and/or
b) the subject is fed with a mother's milk lacking A-tetrasaccharide and/or at least one LNFP, especially LNFP-I, or with a mother's milk comprising A-tetrasaccharide and/or at least one LNFP, especially LNFP-I, at a concentration of 100 mg/ml or less, preferably 50 mg/mL or less, more preferably 25 mg/mL or less.

In some embodiments, the at least one LNFP is present in a total amount of from 25 mg/L to 5000 mg/L of the nutritional composition or of from 0.02-3.75 g/100 g of the nutritional composition.

In some embodiments, LNFP-I is present in a total amount of from 25 mg/L to 5000 mg/L of the nutritional composition or of from 0.02-3.75 g/100 g of the nutritional composition.

In some embodiments, LNFP-I, LNFP-I and LNFP-III are present in a total amount of from 25 mg/L to 5000 mg/L of the nutritional composition or of from 0.02-3.75 g/100 g of the nutritional composition.

In some embodiments, the subject is an infant, a young child or a child.

In some embodiments, said nutritional composition is an infant formula, a starter infant formula, a follow-on or follow-up infant formula, a baby food, an infant cereal composition, a growing-up milk, a fortifier or a supplement.

In a further aspect, the invention provides a method of improving brain development and/or cognitive development in a subject comprising:
(i) obtaining the mother's milk of said subject;
(ii) analysing the milk for the presence of A-tetrasacharide and/or at least one LNFP, especially LNFP-I;
(iii) if A-tetrasacharide and/or at least one LNFP, especially LNFP-I, is absent or at least one LNFP, especially LNFP-I, is present at a concentration of 100 mg/mL or less, preferably 50 mg/mL or less, more preferably 25 mg/mL or less from the mother's milk, supplementing the milk with a nutritional composition comprising at least one LNFP, especially LNFP-I; and
(iv) administering the supplemented milk to the subject,
wherein the subject is an infant, a young child or a child, and
wherein the improvement in brain development and/or cognitive development comprises or consists of an improvement in language development.

In some embodiments, the at least one LNFP is lacto-N-fucopentaose-I (LNFP-I), lacto-N-fucopentaose-II (LNFP-II), lacto-N-fucopentaose-III (LNFP-III), lacto-N-fucopentaose-V (LNFP-V) or any combination thereof. Suitably, the at least one LNFP is LNFP-I. Suitably, the at least one LNFP is a combination of LNFP-I and LNFP-II. Suitably, the at least one LNFP is a combination of LNFP-I and LNFP-III. Suitably, the at least one LNFP is a combination of LNFP-I, LNFP-II and LNFP-III.

In some embodiments, the nutritional composition further comprises B. kashiwanohense.

In a further aspect, the invention provides a method of improving brain development and/or cognitive development in a subject comprising:
(i) obtaining the mother's milk of said subject;
(ii) analysing the milk for the presence of A-tetrasacharide and/or at least one LNFP, especially LNFP-I;
(iii) if A-tetrasacharide and/or at least one LNFP, especially LNFP-I, is absent or at least one LNFP, especially LNFP-I, is present at a concentration of 100 mg/mL or less, preferably 50 mg/mL or less, more preferably 25 mg/mL or less from the mother's milk, supplementing the milk with at least one LNFP, especially LNFP-I, and optionally with B. kashiwanohense; and
(iv) administering the supplemented milk to the subject, wherein the subject is an infant, a young child or a child, and wherein the improvement in brain development and/or cognitive development comprises or consists of an improvement in language development.

In some embodiments, the at least one LNFP is lacto-N-fucopentaose-I (LNFP-I), lacto-N-fucopentaose-II (LNFP-II), lacto-N-fucopentaose-III (LNFP-III), lacto-N-fucopentaose-V (LNFP-V) or any combination thereof. Suitably, the at least one LNFP is LNFP-I. Suitably, the at least one LNFP is a combination of LNFP-I and LNFP-II. Suitably, the at least one LNFP is a combination of LNFP-I and LNFP-III. Suitably, the at least one LNFP is a combination of LNFP-I, LNFP-II and LNFP-III.

In some embodiments, the improvement in language development comprises or consists of an improvement in receptive language and/or expressive language based on a score of Mullen sub-scale or of the Bayley sub-scale.

In some embodiments, the improvement in language development comprises or consists of an improvement in expressive language based on a score of Mullen sub-scale or of the Bayley sub-scale.

In some preferred embodiments, the improvement in language development is an improvement in expressive language based on a score of Mullen sub-scale or of the Bayley sub-scale.

In some embodiments, the method is of improving brain development and/or cognitive development in a subject by increasing the abundance of Bifidobacterium kashiwanohense in the gastrointestinal tract of the subject.

In a further aspect, the invention provides a method of preparing a nutritional composition comprising:
(i) obtaining the mother's milk of a subject;
(ii) analysing the milk for the presence of A-tetrasacharide and/or at least one LNFP, especially LNFP-I; and
(iii) if A-tetrasacharide and/or LNFP-I is absent or at least one LNFP, especially LNFP-I, is present at a concentration of 100 mg/mL or less, preferably 50 mg/ml or less, more preferably 25 mg/mL or less from the mother's milk, supplementing the milk with a nutritional composition comprising at least one LNFP, especially LNFP-I, and optionally with B. kashiwanohense, wherein the subject is an infant, a young child or a child.

In some embodiments, the at least one LNFP is lacto-N-fucopentaose-I (LNFP-I), lacto-N-fucopentaose-II (LNFP-II), lacto-N-fucopentaose-III (LNFP-III), lacto-N-fucopentaose-V (LNFP-V) or any combination thereof. Suitably, the at least one LNFP is LNFP-I. Suitably, the at least one LNFP is a combination of LNFP-I and LNFP-II. Suitably, the at least one LNFP is a combination of LNFP-I and LNFP-III. Suitably, the at least one LNFP is a combination of LNFP-I, LNFP-II and LNFP-III.

In a further aspect, the invention provides a method of preparing a nutritional composition for improving brain development and/or cognitive development in a subject comprising:
(i) obtaining the mother's milk of said subject;
(ii) analysing the milk for the presence of A-tetrasacharide and/or at least one LNFP, especially LNFP-I; and
(iii) if A-tetrasacharide and/or at least one LNFP, especially LNFP-I, is absent or at least one LNFP, especially LNFP-I, is present at a concentration of 100 mg/mL or less, preferably 50 mg/mL or less, more preferably 25 mg/mL or less from the mother's milk, supplementing the milk with a nutritional composition comprising at least one LNFP, especially LNFP-I, and optionally with B. kashiwanohense,
wherein the subject is an infant, a young child or a child, and
wherein the improvement in brain development and/or cognitive development comprises or consists of an improvement in language development or an improvement in fine motor skills.

Suitably, the improvement in brain development and/or cognitive development comprises or consists of an improvement in language development.

Suitably, the improvement in brain development and/or cognitive development comprises or consists of an improvement in fine motor skills or an improvement in fine motor skills.

In some embodiments, the at least one LNFP is lacto-N-fucopentaose-I (LNFP-I), lacto-N-fucopentaose-II (LNFP-II), lacto-N-fucopentaose-III (LNFP-III), lacto-N-fucopentaose-V (LNFP-V) or any combination thereof. Suitably, the at least one LNFP is LNFP-I. Suitably, the at least one LNFP is a combination of LNFP-I and LNFP-II. Suitably, the at least one LNFP is a combination of LNFP-I and LNFP-III. Suitably, the at least one LNFP is a combination of LNFP-I, LNFP-II and LNFP-III.

In a further aspect, the invention provides a nutritional composition comprising at least one LNFP, especially lacto-N-fucopentaose-I (LNFP-I), and Bifidobacterium kashiwanohense.

In some embodiments, the at least one LNFP is lacto-N-fucopentaose-I (LNFP-I), lacto-N-fucopentaose-II (LNFP-II), lacto-N-fucopentaose-III (LNFP-III), lacto-N-fucopentaose-V (LNFP-V) or any combination thereof. Suitably, the at least one LNFP is LNFP-I. Suitably, the at least one LNFP is a combination of LNFP-I and LNFP-II. Suitably, the at least one LNFP is a combination of LNFP-I and LNFP-III. Suitably, the at least one LNFP is a combination of LNFP-I, LNFP-II and LNFP-III.

In a further aspect, the invention provides a combination comprising or consisting of at least one LNFP, especially lacto-N-fucopentaose-I (LNFP-I), and Bifidobacterium kashiwanohense. Suitably, the combination consists of LNFP-I and Bifidobacterium kashiwanohense.

In some embodiments, the at least one LNFP is lacto-N-fucopentaose-I (LNFP-I), lacto-N-fucopentaose-II (LNFP-II), lacto-N-fucopentaose-III (LNFP-III), lacto-N-fucopentaose-V (LNFP-V) or any combination thereof. Suitably, the at least one LNFP is LNFP-I. Suitably, the at least one LNFP is a combination of LNFP-I and LNFP-II. Suitably, the at least one LNFP is a combination of LNFP-I and LNFP-III. Suitably, the at least one LNFP is a combination of LNFP-I, LNFP-II and LNFP-III.

In a further aspect, the invention provides a nutritional composition comprising a combination of lacto-N-fucopentaose-I (LNFP-I), lacto-N-fucopentaose-II (LNFP-II), lacto-N-fucopentaose-III (LNFP-III) and Bifidobacterium kashiwanohense.

In a further aspect, the invention provides a combination comprising or consisting of lacto-N-fucopentaose-I (LNFP-I), lacto-N-fucopentaose-II (LNFP-II), lacto-N-fucopentaose-III (LNFP-III), and Bifidobacterium kashiwanohense. Suitably, the combination consists of LNFP-I and Bifidobacterium kashiwanohense. Suitably, the combination consists of LNFP-I, LNFP-II, LNFP-III and Bifidobacterium kashiwanohense.

In some embodiments, the at least one LNFP is present in a total amount of from 25 mg/L to 5000 mg/L of the nutritional composition or of from 0.02-3.75 g/100 g of the nutritional composition.

In some embodiments, LNFP-I is present in a total amount of from 25 mg/L to 5000 mg/L of the nutritional composition or of from 0.02-3.75 g/100 g of the nutritional composition.

In some embodiments, LNFP-I, LNFP-I and LNFP-III are present in a total amount of from 25 mg/L to 5000 mg/L of the nutritional composition or of from 0.02-3.75 g/100 g of the nutritional composition.

In some embodiments, said nutritional composition is an infant formula, a starter infant formula, a follow-on or follow-up infant formula, a baby food, an infant cereal composition, a growing-up milk, a fortifier or a supplement.

### BRIEF DESCRIPTION OF THE FIGURES

FIG. 1: Scatter plot of the age-regressed value for sensorimotor functional network strength and LNFP-I breastmilk levels in A-tetra+ subjects. The lines represent the linear regression for each quartile of the population (based on the LNFP-I levels).

FIG. 2: Scatter plot of the age-regressed value for auditory functional network strength and LNFP-I breastmilk levels in A-tetra+ subjects. The lines represent the linear regression for each quartile of the population (based on the LNFP-I levels).

FIG. 3: Scatter plot of the age-regressed value for fine motor scores on Bayley-III Scale of Infant and Toddler Development at 6 months (p=0.012) and LNFP-I, II and Ill breastmilk levels collected from mother's milk at 3 months. The line represents the linear regression for each quartile of the population (based on the LNFP-I, -II and -III levels).

FIG. 4: Scatter plot of the age-regressed value for cognition score on Bayley-III Scale at 6 months (p=0.074) and LNFP-I, -II and -III breastmilk levels collected from mother's milk at 3 months. The line represents the linear regression for each quartile of the population (based on the LNFP-I, -II and -III levels).

### DETAILED DESCRIPTION OF THE INVENTION

### Definitions

As used herein, the following terms have the following meanings.

The term “subject” refers to an infant, young child, small for gestational age (SGA) or a preterm infant.

The term “infant” means a child under the age of 12 months.

The expression “young child” means a child aged between one and three years, also called toddler.

The term “child” means a child aged between three and twelve years. Preferably, the term “child” means a child aged between three and six years.

A “preterm” or “premature” subject means an infant or young child who was not born at term. Generally, it refers to an infant or young child born prior 36 weeks of gestation.

By the expression “small for gestational age” or “SGA” it is referred to an infant or young child who is smaller in size than normal for their gestational age at birth, most commonly defined as a birthweight below the 10th percentile for the gestational age. In some embodiments, SGA may be associated with intrauterine growth restriction (IUGR), which refers to a condition in which a foetus is unable to achieve its potential size.

The expression “nutritional composition” means a composition which nourishes a subject. This nutritional composition is usually to be taken orally or intravenously. It may include a lipid or fat source, a carbohydrate source and/or a protein source. In a particular embodiment the nutritional composition is a ready-to-drink composition such as a ready-to-drink formula.

In a particular embodiment, the nutritional composition of the present invention is a “synthetic nutritional composition”. The expression “synthetic nutritional composition” means a mixture obtained by chemical and/or biological means, which can be chemically identical to the mixture naturally occurring in mammalian milks (i.e. the synthetic nutritional composition is not breast milk).

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose).

The expression “infant formula” encompasses both “starter infant formula” and “follow-up formula” or “follow-on formula”.

A “follow-up formula” or “follow-on formula” is given from the 6th month onwards and includes “growing-up milk”. It constitutes the principal liquid element in the progressively diversified diet of this category of person.

The expression “growing-up milk” (or “GUM”) refers to a milk-based drink generally with added vitamins and minerals, that is intended for young children or children.

The expression “baby food” means a foodstuff intended for particular nutritional use by infants or young children during the first years of life.

The expression “infant cereal composition” means a foodstuff intended for particular nutritional use by infants or young children during the first years of life.

The term “fortifier” refers to liquid or solid nutritional compositions suitable for mixing with breast milk or infant formula.

The expression “weaning period” means the period during which the mother's milk is substituted by other food in the diet of an infant or young child.

The “mother's milk” should be understood as the breast milk including colostrum (first milk), transitional or mature milk of the mother.

An “oligosaccharide” is a saccharide polymer containing a small number (typically three to ten) of simple sugars (monosaccharides).

The term “HMO” or “HMOs” refers to human milk oligosaccharide(s). These carbohydrates are resistant to enzymatic hydrolysis by digestive enzymes (e.g. pancreatic and/or brush border), indicating that they may display functions not directly related to their caloric value. It has especially been illustrated that they play a vital role in the early development of infants and young children, such as the maturation of the immune system. Many different kinds of HMOs are found in the human milk. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk-over 130 such structures have been identified so far. Almost all of them have a lactose moiety at their reducing end while sialic acid and/or fucose (when present) occupy terminal positions at the non-reducing ends. The HMOs can be acidic (e.g. charged sialic acid containing oligosaccharide) or neutral (e.g. fucosylated oligosaccharide). Some examples of HMOs are the fucosylated oligosaccharides, the N-acetylated oligosaccharides and/or the sialylated oligosaccharides.

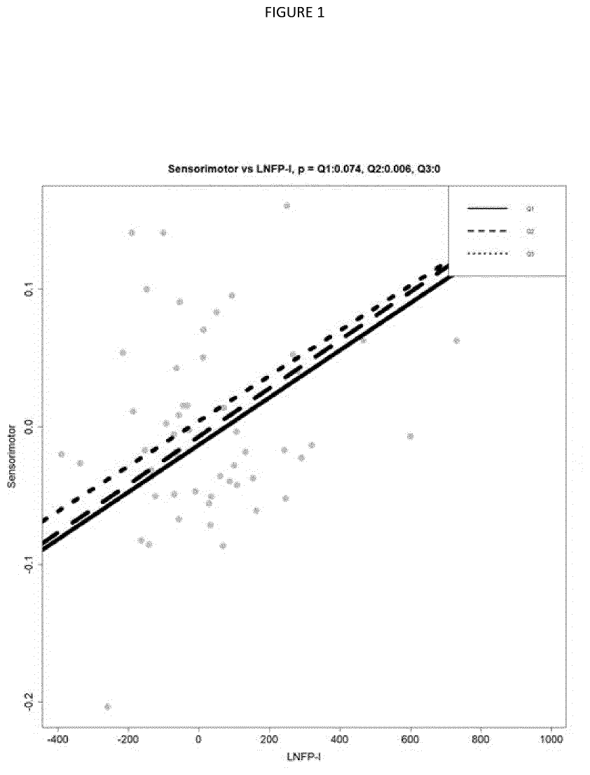

A “fucosylated oligosaccharide” is an oligosaccharide having a fucose residue. It has a neutral nature. Some examples are lacto-N-fucopentaose(s) (LNFP) such as LNFP-I (lacto-N-fucopentaose I), LNFP-II (lacto-N-fucopentaose II), LNFP-III (lacto-N-fucopentaose III), LNFP-V (lacto-N-fucopentaose V), 2′-FL (2′ fucosyllactose), 3-FL (3-fucosyllactose). The structure and relationship between LNFP-I (lacto-N-fucopentaose I), LNFP-II (lacto-N-fucopentaose II), LNFP-III (lacto-N-fucopentaose III) and LNFP-V (lacto-N-fucopentaose V) is described in FIG. 1 of Sprenger et. al., Biology of human milk oligosaccharides: From basic science to clinical evidence, J Hum Nutr Diet. 2022 April; 35 (2): 280-299. doi: 10.1111/jhn. 12990.

A “sialylated oligosaccharide” is a charged sialic acid containing oligosaccharide, i.e. an oligosaccharide having a sialic acid residue. It has an acidic nature. Some examples are 3-SL (3′-sialyllactose) and 6-SL (6′-sialyllactose). The expressions “sialylated oligosaccharide” and “sialyllactose (SL)” can be used interchangeably. The trisaccharide sialyllactose consists of lactose at the reducing terminus and one sialic acid residue at the non-reducing end via an alpha-2,3 binding or alpha-2,6 binding, resulting in 3′-SL and 6′-SL, respectively.

A “precursor of HMO” is a key compound that intervenes in the manufacture of HMO, such as sialic acid and/or fucose.

The nutritional composition of the present invention can be in solid form (e.g. powder) or in liquid form. The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or a follow-on/follow-up formula or a growing-up milk or an infant cereal product or any other formulation designed for infant nutrition).

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10). The microbial cells are generally bacteria or yeasts. In the context of the invention the probiotic is B. kashiwanohense.

The term “cfu” should be understood as colony-forming unit.

The expression “A-tetrasaccharide negative” refers to a subset population of mothers who do not secrete detectable levels of A-tetrasaccharide in their milk. In the context of the invention, “A-tetrasaccharide negative mother”, “A-tetrasacharide is absent” in mother's milk, or “mother's milk lacking A-tetrasaccharide” means that A-tetrasaccharide in mother's milk is present below the limit of detection of 10 mg/L, preferably below 4.4 mg/L.

The structure of “A-tetrasaccharide” is a-D-GalNAc-(I-3)-[alpha-L-Fuc-(I-2)]-D-Gal-(1\hat{ }4)-D-Glc
Wherein
GalNAc=N-acetylgalactosamine
Fuc=Fucose
Gal=Galactose
Glc=Glucose

The term “brain development” refers to the hierarchical process of wiring the brain, such that later development depends on early development, that begins around 2 weeks after conception and continues into young adulthood 20 years later. Brain development builds on itself, as connections eventually link with each other in more complex ways, enabling the child to move and speak and think in ever more complex ways. An example of the hierarchical process of brain development is that language development depends critically on sensory and perceptual development (e.g., discrimination of speech sounds). The term “brain development” comprises the development of (i) auditory brain networks, (ii) sensorimotor brain networks and (iii) default mode brain networks.

The term “cognitive development” refers to the process by which a subject acquires, organizes, and learns to use knowledge. The term “cognitive development” comprises development in terms of (i) information processing, (ii) conceptual resources, (iii) perceptual skill and (iv) language.

The term “language development” comprises two parts (i) receptive language and (ii) expressive language and is based on a score of Mullen sub-scale or of the Bayley sub-scale, for example. Any suitable assessment method for language development, and in particular receptive language and expressive language, known in the art may be employed in the practice of the present invention.

The Mullen Scales of Early Learning (MSEL; Mullen EM. Mullen scales of early learning. AGS Circle Pines, MN, 1995) provides a standardized assessment of language, motor, and perceptual abilities for children of all ability levels through 5 years of age. The revised and updated version yields age-normed t scores, age equivalent scores, and percentile rankings for 5 subdomains: 1) gross motor, 2) fine motor, 3) visual reception, 4) receptive language, and 5) expressive language. Scores from the fine motor, visual reception, receptive language, and expressive language domains can be aggregated to yield an Early Learning Composite or developmental quotient value. It is also common to derive verbal (receptive language age equivalent score+expressive language age equivalent score/chronological age*100) and nonverbal (fine motor age equivalent score+visual reception age equivalent score/chronological age*100) developmental quotient scores from this assessment. The assessment takes between 20 and 45 min, depending on the age of the child.

The term “receptive language” refers to a measure of a child's ability to process linguistic input as the key function, which includes auditory comprehension and auditory sequencing.

The term “expressive language” refers to a measure of a child's ability to use language productively, which includes speaking, language formation and verbal conceptualization.

All percentages are by weight unless otherwise stated.

All weights expressed in g per 100 g of composition are dry weight unless otherwise stated.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

Any reference to prior art documents in this specification is not to be considered an admission that such prior art is widely known or forms part of the common general knowledge in the field.

The invention will now be described in further details. It is noted that the various aspects, features, examples and embodiments described in the present application may be compatible and/or combined together any combination thereof.

Improving Brain Development and/or Cognitive Development

Over the past few decades, studies have investigated the effects of HMOs and the gut microbiome independently on the host's health. For example, numerous animal and clinical studies have supported the potential relationships between HMOs or the gut microbiome independently and the host's cognition and health (such as obesity and cardiovascular disease). In addition, associations between HMOs and gut microbiome species (such as Bifidobacterium species, many of which are well-known consumers of HMOs as prebiotics), have been widely studied to gain insight into how different HMOs may alter infant gut microbiota composition and function.

Collectively, it is highly plausible that a triad relation exists among gut microbiota, HMOs, and cognition during early infancy. That is, both gut microbiota and HMOs could independently and/or through the interactions of the two yield associations with cognitive development in infants. The present inventors have surprisingly found that the HMOs LNFP, especially LNFP-I, are significantly associated with brain and cognitive development and also that the interactions between at least one LNFP, especially LNFP-I, and species of the Bifidobacterium genus (and in particular Bifidobacterium kashiwanohense) are significantly associated with brain and cognitive development. The present inventors have surprisingly found that the HMOs LNFP-I, LNFP-II and LNFP-III are significantly associated with brain and cognitive development such as fine motor skills.

Accordingly, in a first aspect, the invention provides a nutritional composition comprising at least one LNFP, especially LNFP-I, for use in improving brain development and/or cognitive development in a subject.

In a further aspect, the invention provides at least one LNFP, especially LNFP-I, for use in improving brain development and/or cognitive development in a subject.

In a further aspect, the invention provides the use of at least one LNFP, especially LNFP-I, in the manufacture of a medicament for improving brain development and/or cognitive development in a subject. Suitably, the medicament is a nutritional composition as described herein.

In a further aspect, the invention provides the use of a nutritional composition comprising at least one LNFP, especially LNFP-I, for improving brain development and/or cognitive development in a subject.

In a further aspect, the invention provides the use of at least one LNFP, especially LNFP-I, for improving brain development and/or cognitive development in a subject.

In a further aspect, the invention provides a method of improving brain development and/or cognitive development in a subject, the method comprising administering a nutritional composition comprising at least one LNFP, especially LNFP-I, to the subject.

In a further aspect, the invention provides a method of improving brain development and/or cognitive development in a subject, the method comprising administering at least one LNFP, especially LNFP-I, to the subject.

In a further aspect, the invention provides a method of improving brain development and/or cognitive development in a subject, the method comprising administering at least one LNFP, especially lacto-N-fucopentaose-I (LNFP-I), to the subject.

In some embodiments, the at least one LNFP is lacto-N-fucopentaose-I (LNFP-I), lacto-N-fucopentaose-II (LNFP-II), lacto-N-fucopentaose-III (LNFP-III), lacto-N-fucopentaose-V (LNFP-V) or any combination thereof.

In some preferred embodiments, the at least one LNFP is LNFP-I.

In some embodiments, the at least one LNFP is a combination of LNFP-I and LNFP-II.

In some embodiments, the at least one LNFP is a combination of LNFP-I and LNFP-III.

In some preferred embodiments, the at least one LNFP is a combination of LNFP-I, LNFP-II and LNFP-III.

Accordingly, in a further aspect, the invention provides a nutritional composition comprising a combination of lacto-N-fucopentaose-I (LNFP-I), lacto-N-fucopentaose-II (LNFP-II) and lacto-N-fucopentaose-III (LNFP-III), for use in improving brain development and/or cognitive development in a subject.

In a further aspect, the invention provides a combination of lacto-N-fucopentaose-I (LNFP-I), lacto-N-fucopentaose-II (LNFP-II) and lacto-N-fucopentaose-III (LNFP-III), for use in improving brain development and/or cognitive development in a subject.

In a further aspect, the invention provides the use of a nutritional composition comprising a combination of lacto-N-fucopentaose-I (LNFP-I), lacto-N-fucopentaose-II (LNFP-II) and lacto-N-fucopentaose-III (LNFP-III), for improving brain development and/or cognitive development in a subject.

In a further aspect, the invention provides the use of a combination of lacto-N-fucopentaose-I (LNFP-I), lacto-N-fucopentaose-II (LNFP-II) and lacto-N-fucopentaose-III (LNFP-III) for improving brain development and/or cognitive development in a subject.

In a further aspect, the invention provides a method of improving brain development and/or cognitive development in a subject, the method comprising administering a nutritional composition comprising a combination of lacto-N-fucopentaose-I (LNFP-I), lacto-N-fucopentaose-II (LNFP-II) and lacto-N-fucopentaose-III (LNFP-III), to the subject.

In a further aspect, the invention provides a method of improving brain development and/or cognitive development in a subject, the method comprising administering a combination of lacto-N-fucopentaose-I (LNFP-I), lacto-N-fucopentaose-II (LNFP-II) and lacto-N-fucopentaose-III (LNFP-III) to the subject.

In some embodiments, brain development and cognitive development are improved. In some embodiments, brain development is improved. In some embodiments, cognitive development is improved.

In some embodiments, the improvement in brain development and/or cognitive development comprises or consists of an improvement in the development of auditory brain networks, sensorimotor brain networks, default mode brain networks, language, fine motor skills, or combinations thereof.

In some embodiments, the improvement in brain development and/or cognitive development consists of an improvement in the development of auditory brain networks, sensorimotor brain networks, default mode brain networks, language, fine motor skills, or combinations thereof.

In some embodiments, the improvement in brain development and/or cognitive development comprises or consists of an improvement in the development of auditory brain networks, sensorimotor brain networks, default mode brain networks, or combinations thereof.

In some embodiments, the improvement in brain development comprises or consists of an improvement in the development of auditory brain networks, sensorimotor brain networks, default mode brain networks, or combinations thereof.

In some embodiments, the improvement in brain development and/or cognitive development comprises or consists of an improvement in the development of language.

In some embodiments, the improvement in cognitive development comprises or consists of an improvement in the development of language.

In some embodiments, the improvement in language development comprises or consists of an improvement in receptive language and/or expressive language.

In some embodiments, the improvement in brain development and/or cognitive development comprises or consists of an improvement in the development of sensorimotor brain networks.

In some embodiments, the improvement in cognitive development comprises or consists of an improvement in the development of sensorimotor brain networks.

In some embodiments, the improvement in brain development and/or cognitive development comprises or consists of an improvement in the development of fine motor skills.

In some embodiments, the improvement in cognitive development comprises or consists of an improvement in the development of fine motor skills.

Any suitable assessment method for language development, and in particular receptive language and expressive language, known in the art may be employed in the practice of the present invention. Any suitable assessment method for motor development, and in particular fine motor skills, known in the art may be employed in the practice of the present invention.

Suitably, the Mullen Scales of Early Learning (MSEL; Mullen EM. Mullen scales of early learning. AGS Circle Pines, MN, 1995), which provides a standardized assessment of language, motor, and perceptual abilities for children of all ability levels up to 5 years of age, may be used. The MSEL is described in more detail elsewhere herein.

Suitably, the Bayley Scale of Infant and Toddler Development (BSID; Bayley N. Third Edition: Technical Manual. San Antonio, TX: Harcourt Assessment 2006, also called Bayley-III), which provides a standardized assessment of adaptive behaviour, cognition, language, motor and social-emotional abilities for children of all ability levels up to 3.5 years of age, may be used.

Accordingly, in some embodiments, the improvement in language development comprises or consists of an improvement in receptive language and/or expressive language based on a score of Mullen sub-scale or of the Bayley sub-scale.

In some embodiments, the improvement in language development consists of an improvement in receptive language and/or expressive language based on a score of Mullen sub-scale or of the Bayley sub-scale.

In some embodiments, the improvement in language development comprises or consists of an improvement in expressive language based on a score of Mullen sub-scale or of the Bayley sub-scale.

In some embodiments, the improvement in brain development and/or cognitive development comprises or consists of an improvement in expressive language based on a score of Mullen sub-scale or of the Bayley sub-scale.

In some embodiments, improvement in receptive language and/or expressive language is based on a score of Mullen sub-scale.

In some embodiments, improvement in receptive language and/or expressive language is based on a score of the Bayley sub-scale.

Accordingly, in some embodiments, the improvement in fine motor skills is based on a score of Mullen sub-scale or of the Bayley sub-scale.

In some embodiments, the improvement in brain development and/or cognitive development comprises or consists of an improvement in fine motor skills based on a score of Mullen sub-scale or of the Bayley sub-scale.

In some embodiments, improvement in fine motor skills is based on a score of Mullen sub-scale.

In some embodiments, improvement in fine motor skills is based on a score of the Bayley sub-scale

The determination of an improvement in brain development and/or cognitive development may be carried out using any method known in the art. For example, the method disclosed by Smith et al. (2009) may be employed (Smith et al., PNAS, 2009, 106:13040-13045). By way of further example, the methods employed herein may be used (see Materials & Methods, Example 1 and Example 2).

Suitably, brain development and/or cognitive development are improved in a subject using a nutritional composition according to the invention, a combination according to the invention, LNFP-I, or LNFP-I in combination with B. kashiwanohense, when compared to a corresponding nutritional composition which does not comprise at least one LNFP (such as LNFP-I) or which does not comprise LNFP-I in combination with B. kashiwanohense.

Suitably, the nutritional composition is for use in improving brain development and/or cognitive development in a subject by increasing the abundance of Bifidobacterium kashiwanohense in the gastrointestinal tract of the subject.

Suitably, the combination is for use in improving brain development and/or cognitive development in a subject by increasing the abundance of Bifidobacterium kashiwanohense in the gastrointestinal tract of the subject.

In some preferred embodiments, the at least one LNFP, especially LNFP-I, may be used in combination with B. kashiwanohense.

In some preferred embodiments, the nutritional composition further comprises B. kashiwanohense.

Accordingly, in a further aspect, the invention provides a nutritional composition comprising at least one LNFP, especially LNFP-I, and B. kashiwanohense for use in improving brain development and/or cognitive development in a subject.

In a further aspect, the invention provides a combination of at least one LNFP, especially LNFP-I, and B. kashiwanohense for use in improving brain development and/or cognitive development in a subject.

In a further aspect, the invention provides the use of a combination of at least one LNFP, especially LNFP-I, and B. kashiwanohense in the manufacture of a medicament for improving brain development and/or cognitive development in a subject. Suitably, the medicament is a nutritional composition as described herein.

In a further aspect, the invention provides the use of a nutritional composition comprising at least one LNFP, especially LNFP-I, and B. kashiwanohense for improving brain development and/or cognitive development in a subject.

In a further aspect, the invention provides the use of a combination of at least one LNFP, especially LNFP-I, and B. kashiwanohense for improving brain development and/or cognitive development in a subject.

In a further aspect, the invention provides a method of improving brain development and/or cognitive development in a subject, the method comprising administering a nutritional composition comprising at least one LNFP, especially LNFP-I, and B. kashiwanohense to the subject.

In a further aspect, the invention provides a method of improving brain development and/or cognitive development in a subject, the method comprising administering a combination of at least one LNFP, especially LNFP-I, and B. kashiwanohense to the subject.

The at least one LNFP may be at least one LNFP as described herein. Accordingly, in a further aspect, the invention provides a nutritional composition comprising a combination of lacto-N-fucopentaose-I (LNFP-I), lacto-N-fucopentaose-II (LNFP-II), lacto-N-fucopentaose-III (LNFP-III), and B. kashiwanohense for use in improving brain development and/or cognitive development in a subject.

In a further aspect, the invention provides comprising a combination of lacto-N-fucopentaose-I (LNFP-I), lacto-N-fucopentaose-II (LNFP-II), lacto-N-fucopentaose-III (LNFP-III), and B. kashiwanohense for use in improving brain development and/or cognitive development in a subject.

In a further aspect, the invention provides the use of a combination of lacto-N-fucopentaose-I (LNFP-I), lacto-N-fucopentaose-II (LNFP-II), lacto-N-fucopentaose-III (LNFP-III), and B. kashiwanohense in the manufacture of a medicament for improving brain development and/or cognitive development in a subject. Suitably, the medicament is a nutritional composition as described herein.

In a further aspect, the invention provides the use of a nutritional composition comprising a combination of lacto-N-fucopentaose-I (LNFP-I), lacto-N-fucopentaose-II (LNFP-II), lacto-N-fucopentaose-III (LNFP-III), and B. kashiwanohense for improving brain development and/or cognitive development in a subject.

In a further aspect, the invention provides the use of a combination of lacto-N-fucopentaose-I (LNFP-I), lacto-N-fucopentaose-II (LNFP-II), lacto-N-fucopentaose-III (LNFP-III), and B. kashiwanohense for improving brain development and/or cognitive development in a subject.

In a further aspect, the invention provides a method of improving brain development and/or cognitive development in a subject, the method comprising administering a nutritional composition comprising a combination of lacto-N-fucopentaose-I (LNFP-I), lacto-N-fucopentaose-II (LNFP-II), lacto-N-fucopentaose-III (LNFP-III), and B. kashiwanohense to the subject.

In a further aspect, the invention provides a method of improving brain development and/or cognitive development in a subject, the method comprising administering a combination of lacto-N-fucopentaose-I (LNFP-I), lacto-N-fucopentaose-II (LNFP-II), lacto-N-fucopentaose-III (LNFP-III), and B. kashiwanohense to the subject.

In a further aspect, the invention provides a nutritional composition comprising at least one LNFP, especially lacto-N-fucopentaose-I (LNFP-I), and Bifidobacterium kashiwanohense.

In a further aspect, the invention provides a combination comprising or consisting of at least one LNFP, especially lacto-N-fucopentaose-I (LNFP-I) and Bifidobacterium kashiwanohense. Suitably, the combination consists of at least one LNFP, especially LNFP-I and Bifidobacterium kashiwanohense.

The at least one LNFP may be at least one LNFP as described herein.

Accordingly, in a further aspect, the invention provides a nutritional composition comprising a combination of lacto-N-fucopentaose-I (LNFP-I), lacto-N-fucopentaose-II (LNFP-II), lacto-N-fucopentaose-III (LNFP-III) and Bifidobacterium kashiwanohense.

In a further aspect, the invention provides a combination comprising or consisting of lacto-N-fucopentaose-I (LNFP-I), lacto-N-fucopentaose-II (LNFP-II), lacto-N-fucopentaose-III (LNFP-III), and Bifidobacterium kashiwanohense. Suitably, the combination consists of LNFP-I and Bifidobacterium kashiwanohense. Suitably, the combination consists of LNFP-I, LNFP-II, LNFP-III and Bifidobacterium kashiwanohense.

The nutritional composition or combination according to the invention may contain from 10e3 to 10e12 cfu of B. kashiwanohense, more preferably between 10e7 and 10e12 cfu such as between 10e8 and 10e10 cfu of B. kashiwanohense per g of composition on a dry weight basis.

B. kashiwanohense was isolated from healthy infant faeces. For example, this bacterium has previously been characterized by determining its phenotypic and biochemical features and phylogenetic positions based on partial 16S rRNA gene sequence analysis (Morita et al., International Journal of Systematic and Evolutionary Microbiology, 2011, 61:2610-2615). The GenBank/EMBL/DDBJ accession numbers for the 16S rRNA and partial hsp60 gene sequences of two strains of B. kashiwanohense are (i) AB491757 and AB578933 and (ii) are AB425276.2 and AB491759.2, respectively. One strain of B. kashiwanohense is publicly available from two collections with the accession numbers JCM 15439 and DSM 21854 (Morita et al., International Journal of Systematic and Evolutionary Microbiology, 2011, 61:2610-2615).

The B. kashiwanohense may be a B. kashiwanohense having at least 99% (suitably, at least 99.9%) Average Nucleotide Identity (ANI) to any B. kashiwanohense known to the skilled person.

As used herein the term “Average nucleotide identity (ANI)” refers to a distance-based approach to delineate species based on pair-wise comparisons of their genome sequences. ANI is an in silico approach for phylogenetic definition of a species and has become the gold standard for species delineation (Goris et al., 2007, Int. J. Syst. Evol. Microbiol. 57:81-91; Kim et al., 2014, Int. J. Syst. Evol. Micr. 64:346-351; Richter et al., 2009, P Natl Acad Sci USA 106:19126-19131; and Chan et al., 2012, Bmc. Microbiol. 12).

The ANI of the shared genes between two strains is known to be a robust means to compare genetic relatedness among strains. Strains with ANI values of at least about 96% can be considered to belong to the same species (Konstantinidis and Tiedje, 2005, Proc Natl Acad Sci USA, 102 (7): 2567-72; and Goris et al., 2007, Int Syst Evol Microbiol. 57 (Pt 1): 81-91), while ANI values of at least about 99% indicate that the bacterial genomes belong to the same strain. The ANI between two bacterial genomes is calculated from pair-wise comparisons of all sequences shared between any two strains and can be determined, for example, using any of a number of publicly available ANI tools, including but not limited to OrthoANI with usearch (Yoon et al., 2017, Antonie van Leeuwenhoek 110:1281-1286); ANI Calculator, JSpecies (Richter and Rossello-Mora, 2009, Proc Natl Acad Sci USA 106:19126-19131); and JSpeciesWS (Richter et al., 2016, Bioinformatics 32:929-931). Other methods for determining the ANI of two genomes are known in the art (Konstantinidis, K. T. and Tiedje, 2005, J. M., Proc. Natl. Acad. Sci. U.S.A., 102:2567-2572; and Varghese et al., 2015, Nucleic Acids Research, 43(14):6761-6771).

Suitably, the B. kashiwanohense has at least 99% (suitably, at least 99.1%, at least 99.2%, at least 99.3%, at least 99.4%, at least 99.5%, at least 99.6%, at least 99.7%, at least 99.8%, at least 99.9%) ANI to B. kashiwanohense JCM 15439 and/or B. kashiwanohense DSM 21854. Preferably, the B. kashiwanohense has at least 99.9% ANI to B. kashiwanohense JCM 15439 and/or B. kashiwanohense DSM 21854.

In a further aspect, the invention provides the use of a nutritional composition comprising at least one LNFP, especially LNFP-I, for increasing the abundance of B. kashiwanohense in the gastrointestinal tract of a subject.

In some embodiments, the invention provides the use of a nutritional composition comprising lacto-N-fucopentaose-I (LNFP-I) for increasing the abundance of B. kashiwanohense in the gastrointestinal tract of a subject.

In a further aspect, the invention provides the use of at least one LNFP, especially LNFP-I, for increasing the abundance of B. kashiwanohense in the gastrointestinal tract of a subject.

In some embodiments, the invention provides the use of lacto-N-fucopentaose-I (LNFP-I) for increasing the abundance of B. kashiwanohense in the gastrointestinal tract of a subject.

In a further aspect, the invention provides the use of a nutritional composition comprising a combination of lacto-N-fucopentaose-I (LNFP-I), lacto-N-fucopentaose-II (LNFP-II) and lacto-N-fucopentaose-III (LNFP-III), for increasing the abundance of B. kashiwanohense in the gastrointestinal tract of a subject.

In a further aspect, the invention provides the use of a combination of lacto-N-fucopentaose-I (LNFP-I), lacto-N-fucopentaose-II (LNFP-II) and lacto-N-fucopentaose-III (LNFP-III), for increasing the abundance of B. kashiwanohense in the gastrointestinal tract of a subject.

Suitably, the relative abundance of B. kashiwanohense in the gastrointestinal tract of the subject is increased. Suitably, the abundance of B. kashiwanohense or relative abundance of B. kashiwanohense is increased in a subject using a nutritional composition according to the invention or the at least one LNFP, especially LNFP-I, when compared to a corresponding nutritional composition which does not comprise the at least one LNFP, especially LNFP-I.

In some embodiments, the at least one LNFP, especially LNFP-I, is present in a total amount of from 25 mg/L to 5000 mg/L of the composition, nutritional composition, or combination according to the invention or of from 0.02 g/100 g to 4 g/100 g of the composition, nutritional composition or combination according to the invention. Suitably, the at least one LNFP, especially LNFP-I, is present in a total amount of from 50 mg/L to 2500 mg/L, for example from 60 mg/L to 2000 mg/L, for example from 80 mg/L to 1000 mg/L of the composition, nutritional composition or combination according to the invention. Suitably, the at least one LNFP, especially LNFP-I, is present in a total amount of from 0.04 g/100 g to 2 g/100 g, for example from 0.05 g/100 g to 1.6 g/100 g, for example from 0.07 g/100 g to 0.8 g/100 g of the composition, nutritional composition or combination (dry weight).

In some embodiments, LNFP-I is present in a total amount of from 25 mg/L to 5000 mg/L of the composition, nutritional composition, or combination according to the invention or of from 0.02 g/100 g to 4 g/100 g of the composition, nutritional composition or combination according to the invention. Suitably, LNFP-I is present in a total amount of from 50 mg/L to 2500 mg/L, for example from 60 mg/L to 2000 mg/L, for example from 80 mg/L to 1000 mg/L of the composition, nutritional composition or combination according to the invention. Suitably, LNFP-I is present in a total amount of from 0.04 g/100 g to 2 g/100 g, for example from 0.05 g/100 g to 1.6 g/100 g, for example from 0.07 g/100 g to 0.8 g/100 g of the composition, nutritional composition or combination (dry weight).

In some embodiments, LNFP-I, LNFP-II, and LNFP-III are present in a total amount of from 25 mg/L to 5000 mg/L of the composition, nutritional composition, or combination according to the invention or of from 0.02 g/100 g to 4 g/100 g of the composition, nutritional composition or combination according to the invention. Suitably, LNFP-I, LNFP-II, and LNFP-III are present in a total amount of from 50 mg/L to 2500 mg/L, for example from 60 mg/L to 2000 mg/L, for example from 80 mg/L to 1000 mg/L of the composition, nutritional composition or combination according to the invention. Suitably, LNFP-I, LNFP-II, and LNFP-III are present in a total amount of from 0.04 g/100 g to 2 g/100 g, for example from 0.05 g/100 g to 1.6 g/100 g, for example from 0.07 g/100 g to 0.8 g/100 g of the composition, nutritional composition or combination (dry weight).

In a further aspect, the invention provides a method of improving brain development and/or cognitive development in a subject comprising:
(i) obtaining the mother's milk of said subject;
(ii) analysing the milk for the presence of A-tetrasacharide and/or at least one LNFP, especially LNFP-I;
(iii) if A-tetrasacharide and/or at least one LNFP, especially LNFP-I is absent or at least one LNFP, especially LNFP-I, is present at a concentration of 100 mg/ml or less, preferably 50 mg/mL or less, more preferably 25 mg/mL or less from the mother's milk, supplementing the milk with a nutritional composition comprising the at least one LNFP, especially LNFP-I; and
(iv) administering the supplemented milk to the subject,
wherein the subject is an infant, a young child or a child, and
wherein the improvement in brain development and/or cognitive development comprises or consists of an improvement in language development or an improvement in fine motor skills.

Preferably, the improvement in brain development and/or cognitive development comprises or consists of an improvement in language development.

Suitably, the improvement in brain development and/or cognitive development comprises or consists of an improvement in fine motor skills or an improvement in fine motor skills.

The at least one LNFP may be at least one LNFP as described herein.

The nutritional composition may be a nutritional composition as described elsewhere herein. Suitably, the nutritional composition further comprises B. kashiwanohense.

In some embodiments, the milk is analysed for the presence of A-tetrasacharide.

In some embodiments, the milk is analysed for the presence of at least one LNFP, especially LNFP-I.

In some embodiments, the milk is analysed for the presence of A-tetrasacharide and at least one LNFP, especially LNFP-I.

The at least one LNFP may be at least one LNFP as described herein.

In a further aspect, the invention provides a method of improving brain development and/or cognitive development in a subject comprising:
(i) obtaining the mother's milk of said subject;
(ii) analysing the milk for the presence of A-tetrasacharide and/or at least one LNFP, especially LNFP-I;
(iii) if A-tetrasacharide and/or at least one LNFP, especially LNFP-I is absent from the mother's milk or at least one LNFP, especially LNFP-I is present at a concentration of 100 mg/ml or less, preferably 50 mg/mL or less, more preferably 25 mg/ml or less in the mother's milk, supplementing the milk with a combination of the at least one LNFP, especially LNFP-1, and B. kashiwanohense; and
(iv) administering the supplemented milk to the subject,
wherein the subject is an infant, a young child or a child, and
wherein the improvement in brain development and/or cognitive development comprises or consists of an improvement in language development or an improvement in fine motor skills.

Preferably, the improvement in brain development and/or cognitive development comprises or consists of an improvement in language development.

Suitably, the improvement in brain development and/or cognitive development comprises or consists of an improvement in fine motor skills or an improvement in fine motor skills.

The at least one LNFP may be at least one LNFP as described herein.

In some embodiments, the milk is analysed for the presence of A-tetrasacharide.

In some embodiments, the milk is analysed for the presence of at least one LNFP, especially LNFP-I.

In some embodiments, the milk is analysed for the presence of A-tetrasacharide and at least one LNFP, especially LNFP-I.

The at least one LNFP may be at least one LNFP as described herein.

In some embodiments, the improvement in language development is as described elsewhere herein.

In some embodiments, the improvement in fine motor skills is as described elsewhere herein.

Some particular embodiments according to the invention are defined in the following clauses 1-15:

Clause 1. A nutritional composition comprising lacto-N-fucopentaose-I (LNFP-I) for use in improving brain development and/or cognitive development in a subject.

Clause 2. The nutritional composition for use according to clause 1, wherein the improvement in brain development and/or cognitive development comprises or consists of an improvement in the development of auditory brain networks, sensorimotor brain networks, default mode brain networks, language, or combinations thereof.

Clause 3. The nutritional composition for use according to clause 2, wherein the improvement in language development comprises or consists of an improvement in receptive language and/or expressive language based on a score of Mullen sub-scale or of the Bayley sub-scale.

Clause 4. The nutritional composition for use according to clause 2 or clause 3, wherein the improvement in language development comprises or consists of an improvement in expressive language based on a score of Mullen sub-scale or of the Bayley sub-scale.

Clause 5. The nutritional composition for use according to any one of the preceding clauses, wherein the improvement in brain development and/or cognitive development comprises or consists of an improvement in expressive language based on a score of Mullen sub-scale or of the Bayley sub-scale.

Clause 6. The nutritional composition for use according to clause 5, wherein the nutritional composition is for use in improving brain development and/or cognitive development in a subject by increasing the abundance of Bifidobacterium kashiwanohense in the gastrointestinal tract of the subject.

Clause 7. The nutritional composition for use according to clause 6, wherein the subject is:
a) born of an A-tetrasaccharide negative mother; and/or
b) the subject is fed with a mother's milk lacking A-tetrasaccharide and/or LNFP-I or with a mother's milk having LNFP-I at a concentration of 100 mg/mL or less, preferably 50 mg/mL or less, more preferably 25 mg/mL or less.

Clause 8. The nutritional composition for use according to any one of the preceding clauses, wherein LNFP-I is present in a total amount of from 25 mg/L to 5000 mg/L of the nutritional composition or of from 0.02-3.75 g/100 g of the nutritional composition.

Clause 9. The nutritional composition for use according to any one of the preceding clauses, wherein the nutritional composition further comprises B. kashiwanohense.

Clause 10. The nutritional composition for use according to any one of the preceding clauses, wherein the subject is an infant, a young child or a child, and/or wherein said nutritional composition is an infant formula, a starter infant formula, a follow-on or follow-up infant formula, a baby food, an infant cereal composition, a growing-up milk, a fortifier or a supplement.

Clause 11. Use of a nutritional composition comprising lacto-N-fucopentaose-I (LNFP-I) for improving brain development and/or cognitive development in a subject.

Clause 12. Use of a nutritional composition comprising lacto-N-fucopentaose-I (LNFP-I) for increasing the abundance of Bifidobacterium kashiwanohense in the gastrointestinal tract of a subject.

Clause 13. A method of improving brain development and/or cognitive development in a subject comprising administering a nutritional composition comprising lacto-N-fucopentaose-I (LNFP-I) to the subject.

Clause 14. A method of improving brain development and/or cognitive development in a subject comprising:
(i) obtaining the mother's milk of said subject;
(ii) analysing the milk for the presence of A-tetrasacharide and/or LNFP-I;
(iii) if A-tetrasacharide and/or LNFP-I is absent or LNFP-I present at a concentration of 100 mg/mL or less, preferably 50 mg/mL or less, more preferably 25 mg/ml or less from the mother's milk, supplementing the milk with a nutritional composition comprising lacto-N-fucopentaose-I (LNFP-I); and
(iv) administering the supplemented milk to the subject,
wherein the subject is an infant, a young child or a child, and
wherein the improvement in brain development and/or cognitive development comprises or consists of an improvement in language development,
optionally, wherein the improvement in language development is an improvement in expressive language based on a score of Mullen sub-scale or of the Bayley sub-scale.

Clause 15. A nutritional composition comprising lacto-N-fucopentaose-I (LNFP-I) and Bifidobacterium kashiwanohense Subject

In a preferred embodiment, the subject is born of an A-tetrasaccharide negative mother and/or the subject is fed with a mother's milk lacking A-tetrasaccharide. Suitably, the subject is born of an A-tetrasaccharide negative mother. Suitably, the subject is fed with a mother's milk lacking A-tetrasaccharide.

An A-tetrasaccharide negative mother may be identified using any method known in the art. Suitably, an A-tetrasaccharide negative mother is a mother whose milk lacks any detectable levels of A-tetrasaccharide. Suitably, an A-tetrasaccharide negative mother may be blood group B or blood group O according to the ABO blood typing system. Suitably, mother's milk lacking A-tetrasaccharide is mother's milk having no detectable A-tetrasaccharide. Levels of A-tetrasaccharide in mother's milk may be determined using any suitable method known in that art. For example, levels of A-tetrasaccharide may be determined as described herein (see Example 2, Materials & Methods).

In some embodiments, the nutritional composition or combination according to the invention is for use in infants, young children or children.

In one embodiment, the subject is an infant. In one embodiment, the subject is a young child. In one embodiment, the subject is a child.

The infants, young children or children may be born term or preterm. In a particular embodiment the nutritional composition or combination of the invention is for use in infants, young children or children that were born preterm. Preterm infants may be at increased risk of poor nutrient utilization, impaired lean body mass growth, fat accumulation in the visceral area and metabolic disease later in life. Thus, in one embodiment the nutritional composition or combination of the invention is for use in preterm infants.

In other embodiments, the subject is a juvenile animal, preferably wherein the animal is a pet. A pet may be an animal selected from dogs, cats, birds, fish, rodents such as mice, rats, and guinea pigs, rabbits, etc. In some embodiments, the pet is a small dog breed.

The term “juvenile” may refer to an individual that has not yet reached adulthood.

The nutritional composition or combination of the present invention may also be used in subject that was born by C-section or that was vaginally delivered.

In some embodiments the nutritional composition or combination according to the invention can be for use before and/or during the weaning period.

In some embodiments the nutritional composition or combination according to the invention is for use in a subject at risk and/or in need.

The subject at risk and/or in need may be bottle-fed and/or formula-fed. The subject at risk and/or in need may be a subject who has difficulties in expressive language.

In one embodiment the composition or combination of the invention is given to the subject as a supplementary composition to the mother's milk. In some embodiments the subject receives the mother's milk during at least the first 2 weeks, first 1, 2, 4, or 6 months. In one embodiment the nutritional composition or combination of the invention is given to the subject after such period of mother's nutrition, or is given together with such period of mother's milk nutrition. In another embodiment the composition or combination is given to the subject as the sole or primary nutritional composition during at least one period of time, e.g. after the 1^{st}, 2^{nd} or 4^{th} month of life, during at least 1, 2, 4 or 6 months. In one embodiment the nutritional composition of the invention is a complete nutritional composition (fulfilling all or most of the nutritional needs of the subject). In another embodiment the nutrition composition or combination of the invention is a supplement or a fortifier intended for example to supplement human milk or to supplement an infant formula or a follow-on formula.

### Other Ingredients

The nutritional composition or combination according to the present invention may also comprise other types of oligosaccharide(s), polysaccharides and/or a fiber(s) and/or a precursor(s) thereof. The other oligosaccharide and/or fiber and/or precursor thereof may be selected from the list comprising human milk oligosaccharides (HMOs), galacto-oligosaccharides (GOS), fructo-oligosaccharides (FOS), xylooligosaccharides (XOS), cello-oligosaccharides (COS), arabinoxylans, arabinans, xylans, inulin, polydextrose, beta-glucans, pectins and any combination thereof and any derived products, like partial hydrolysis products, thereof. They may be in an amount between 0 and 10% by weight of composition. In a particular embodiment, the nutritional composition or the combination can also contain at least one BMO (bovine milk oligosaccharide).

HMOs which may be included in the nutritional composition or combination according to the present invention may be selected from the group consisting of 2-FL (2-fucosyllactose), 3-FL (3-fucosyllactose), Lacto-difucotetraose (LDFT)), lacto-N-fucopentaose II, lacto-N-fucopentaose III, lacto-N-fucopentaose V, lacto-N-fucohexaose, lacto-N-difucohexaose I, fucosyllacto-N-hexaose, fucosyllacto-N-neohexaose, difucosyllacto-N-hexaose 1, difucosyllacto-N-neohexaose II, para-lacto-N-neohexaose (para-LNnH), LNT (lacto-N-tetraose), LNnT (lacto-N-neotetraose), lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose, lacto-N-decaose, 3-SL (3′ sialyllactose), 6-SL (6′ sialyllactose) and any combination thereof. Suitably, the nutritional composition or combination does not comprise A-tetrasaccharide.

In some embodiments, the nutritional composition or combination according to the invention comprises at least one additional HMO. In other embodiments, the nutritional composition or combination according to the present invention is devoid of any further HMOs. Thus, the at one LNFP, especially LNFP-I, may be the only HMO(s) in the nutritional composition or combination of the invention.

In one embodiment, the B. kashiwanohense is viable. In another embodiment, B. kashiwanohense is non-replicating or inactivated. B. kashiwanohense may be both viable and in some other embodiments.

The nutritional composition according to the invention generally contains a protein source. The protein can be in an amount of from 1.6 to 3 g per 100 kcal. In some embodiments, especially when the composition is intended for premature infants, the protein amount can be between 2.4 and 4 g/100 kcal or more than 3.6 g/100 kcal. In some other embodiments the protein amount can be below 2.0 g per 100 kcal, e.g. between 1.8 to 2 g/100 kcal, or in an amount below 1.8 g per 100 kcal.

Protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some advantageous embodiments, the protein source is whey predominant (i.e. more than 50% of proteins are coming from whey proteins, such as 60% or 70%).

The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids. The proteins may be either fully or partially hydrolysed. It may be desirable to supply partially hydrolysed proteins (degree of hydrolysis between 2 and 20%), for example for infants or young children believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

In an embodiment of the invention, at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

In one particular embodiment, the proteins of the nutritional composition are hydrolysed, fully hydrolysed or partially hydrolysed. The degree of hydrolysis (DH) of the protein can be between 8 and 40, or between 20 and 60 or between 20 and 80 or more than 10, 20, 40, 60, 80 or 90.

The protein component can alternatively be replaced by a mixture or synthetic amino acid, for example for preterm or low birth weight infants.

The nutritional composition according to the present invention generally contains a carbohydrate source. This is particularly preferable in the case where the nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates is lactose.

The nutritional composition according to the present invention generally contains a source of lipids. This is particularly relevant if the nutritional composition of the invention is an infant formula. In this case, the lipid source may be any lipid or fat which is suitable for use in infant formulae. Some suitable fat sources include palm oil, structured triglyceride oil, high oleic sunflower oil and high oleic safflower oil, medium-chain-triglyceride oil. The essential fatty acids linoleic and a-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example about 8:1 to about 10:1.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilisers such as soy, lecithin, citric acid esters of mono- and di-glycerides, and the like.

The nutritional composition of the invention may also contain other substances which may have a beneficial effect such as lactoferrin, nucleotides, nucleosides, and the like.

The nutritional composition of the invention may also contain carotenoid(s). In some particular embodiments of the invention, the nutritional composition of the invention does not comprise any carotenoid.

### Nutritional Composition or Combination

The nutritional composition according to the invention can be for example an infant formula, a starter infant formula, a follow-on or follow-up formula, a growing-up milk, a baby food, an infant cereal composition, a fortifier such as a human milk fortifier, a supplement, a pet food, or a pet food supplement. In some particular embodiments, the composition of the invention is an infant formula, a fortifier or a supplement that may be intended for the first 4 or 6 months of age. In a preferred embodiment the nutritional composition of the invention is an infant formula.

The combination according to the invention can be for example formulated as an infant formula, a starter infant formula, a follow-on or follow-up formula, a growing-up milk, a baby food, an infant cereal composition, a fortifier such as a human milk fortifier, a supplement, a pet food, or a pet food supplement.

In some other embodiments the nutritional composition or combination of the present invention is a fortifier. The fortifier can be a breast milk fortifier (e.g. a human milk fortifier) or a formula fortifier such as an infant formula fortifier or a follow-on/follow-up formula fortifier.

When the nutritional composition or combination is a supplement, it can be provided in the form of unit doses. In such cases it is particularly useful to define the amount of oligosaccharides and B. kashiwanohense in terms of daily dose to be administered to the infant, young child or child.

When the nutritional composition or combination is a supplement, it may comprise at least one LNFP, especially LNFP-I, (and optionally B. kashiwanohense) and no other additional nutrient on top of the excipients necessary to obtain a stable nutritional composition.

The nutritional composition or combination of the present invention can be in solid (e.g. powder), liquid or gelatinous form. In a specific embodiment the nutritional composition or combination is a supplement, wherein the supplement is in powder form and provided in a sachet, preferably a sachet with 0.1 to 20 g per sachet, for example 1 to 10 g per sachet, or in the form of a syrup, preferably a syrup with a total solid concentration of 5 to 75 g/100 mL (5 to 75% (w/v)). When the supplement is in powder form, it may comprise a carrier. It is however preferred that the supplement is devoid of a carrier. When the supplement is in the form of a syrup, the components are preferably dissolved or suspended in water acidified with citrate.

In a particular embodiment the nutritional composition or the combination according to the invention is a hypoallergenic composition. In another particular embodiment the composition or combination according to the invention is a hypoallergenic nutritional composition.

The nutritional composition or combination according to the invention may be prepared in any suitable manner. A composition will now be described by way of example.

For example, a formula such as an infant formula may be prepared by blending together the protein source, the carbohydrate source and the fat source in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

The oligosaccharide(s) may be added at this stage, especially if the final product is to have a liquid form. If the final product is to be a powder, they may likewise be added at this stage if desired.

The liquid mixture is then homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. The oligosaccharide(s) may also or alternatively be added at this stage by dry-mixing or by blending them in a syrup form of crystals, along with the probiotic strain, and the mixture is spray-dried or freeze-dried.

If a liquid composition is preferred, the homogenised mixture may be sterilised then aseptically filled into suitable containers or may be first filled into the containers and then retorted.

In another embodiment, the composition or combination of the invention may be a supplement. The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or parenteral administration as well as vitamins, minerals trace elements and other micronutrients in accordance with the recommendations of Government bodies such as the USRDA.

In a further aspect, the invention provides a method of preparing a nutritional composition comprising:
(i) obtaining the mother's milk of a subject;
(ii) analysing the milk for the presence of A-tetrasacharide and/or at least one LNFP, especially LNFP-I; and
(iii) if A-tetrasacharide and/or at least one LNFP, especially LNFP-I, is absent or at least one LNFP, especially LNFP-I, is present at a concentration of 100 mg/ml or less, preferably 50 mg/mL or less, more preferably 25 mg/mL or less from the mother's milk, supplementing the milk with a nutritional composition comprising the at least one LNFP, especially LNFP-I, wherein the subject is an infant, a young child or a child.

The at least one LNFP may be at least one LNFP as described herein.

In some embodiments, the nutritional composition is formulated for improving brain development and/or cognitive development in a subject as described herein.

In some embodiments, the nutritional composition is suitable for improving brain development and/or cognitive development in a subject as described herein.

In a further aspect, the invention provides a method of preparing a nutritional composition for improving brain development and/or cognitive development in a subject comprising:
(i) obtaining the mother's milk of said subject;
(ii) analysing the milk for the presence of A-tetrasacharide and/or at least one LNFP, especially LNFP-I; and
(iii) if A-tetrasacharide and/or at least one LNFP, especially LNFP-I, is absent or at least one LNFP, especially LNFP-I, is present at a concentration of 100 mg/ml or less, preferably 50 mg/mL or less, more preferably 25 mg/mL or less from the mother's milk, supplementing the milk with a nutritional composition comprising the at least one LNFP, especially LNFP-I,
wherein the subject is an infant, a young child or a child, and
wherein the improvement in brain development and/or cognitive development comprises or consists of an improvement in language development or an improvement in fine motor skills.

Preferably, the improvement in brain development and/or cognitive development comprises or consists of an improvement in language development.

Suitably, the improvement in brain development and/or cognitive development comprises or consists of an improvement in fine motor skills or an improvement in fine motor skills.

The at least one LNFP may be at least one LNFP as described herein.

The different embodiments, details and examples previously described in the specification (e.g. related to the types and amounts of oligosaccharide, the nutritional composition, the administration, the targeted population . . . ) also apply to all these other objects.

### EXAMPLES

The following examples illustrate some specific embodiments of the composition for use according to the present invention. The examples are given solely for the purpose of illustration and are not to be construed as limitations of the present invention, as many variations thereof are possible without departing from the spirit of the invention.

### Materials & Methods

### Study Subjects

Parents enrolled in this study provided written informed consent for the participation of both themselves and their infants. The University of North Carolina at Chapel Hill and University of Minnesota Institutional Review Boards approved all study activities. Using site-based research registries, subjects were enrolled from both universities. Local newborn nurseries, institutional centers with research interest on early brain development, local flyers, and university listservs were additionally used for recruitment. The inclusion criteria were: 1) birth at 37-42 weeks of gestational age; 2) appropriate birth weight for gestational age; and 3) no major pregnancy and delivery complications. The exclusion criteria included: 1) adopted child; 2) presence of autism, intellectual disability, schizophrenia, or bipolar disorder related first degree; 3) less than 2 kilograms of birth weight; 4) neonatal hypoxia (10 minute APGAR<5); 5) having illness requiring more than two days of newborn intensive care unit stay; 6) chromosomal or major congenital abnormality; 7) abnormal magnetic resonance in previous MRI; 8) significant developmental delay or medical illness, or significant genetic or medical conditions impacting growth, development, or cognition (including visual/hearing impairment); 9) contraindication in MRI; and 10) maternal pre-eclampsia, HIV status, placental abruption, and alcohol or illicit drug use during pregnancy. Finally, additional inclusion criteria for subjects included in this study were infants younger than 12 months old and exclusively/predominantly breastfed during the first four months of life, defined as the infants who were fed less than 20 g or 4 teaspoons per day of complementary foods/liquids (water, apple juice, etc.) and non-formula.

### Human Milk Collections and Analyses

Human milk (HM) samples were obtained from the right breast using a hospital-grade, electric Medela Symphony breast pump at each visit. To ensure that the collected HM samples represented HM composition at each feeding, the samples were gathered until no more HM was expressed. Additionally, whenever possible, HM samples were standardized to the second feed of the day so that the diurnal variation of HM compositions could be minimized. The weight and volume of the samples were recorded and then vortexed at the highest speed for 2 minutes. A graduated cylinder was used for volume measurement with extra care to avoid bubbles. The total fat content was then measured using mid-infrared spectroscopic analyses (MIRIS Human Milk Analyzer) to ensure that the total fat content, which indicates the quality of milk sampling, was within the expected range of 2.5 mL. Lastly, from the collection bottle, an aliquot of the minimum 30 mL of volume was transferred to a 50 mL polypropylene Falcon tube. Repeat pipette and appropriate tips were used to make eleven aliquots of 1 mL in 1 mL Eppendorf tubes, and nine aliquots of 2 mL in 2 mL Eppendorf tubes for storage in a −80° C. freezer after the collection was done.

For HMO quantifications, a representative 1 mL aliquot of HM was shipped to Neotron Spa (Italy) on dry ice. HMO analyses were done using liquid chromatography with fluorescence detection after labelling with 2-aminobenzamide (Austin S, Benet T., Anal Chim Acta., 2018, 1010:86-96). Using standard curves with authentic HMO standards, the following HMOs were quantified: Lacto-N-fucopentaose-I (LNFP-I), and A-tetra.

### Infant Gut Microbiota Composition and Analyses

Using the Omnigene Gut sample collection kit (DNA GenoTek, Ontario, Canada), stool samples were collected from children's diapers 24 hours prior, during or after the in-person visits. All collected samples, which should be stable up to 60 days in a collection tube, were processed within a week using the following steps. To loosen the fecal samples, they were placed in a dry bead bath. To transfer a fecal sample into the Eppendorf tubes, a sterile transfer pipette was used. Then, between 2×1.5 ml Eppendorf tubes, the fecal sample was evenly split and was frozen and immediately placed in the −80° C. freezer. Finally, all collected fecal samples were shipped to CosmosID Inc. (Germantown, MD, USA) for further analyses detailed below.

### DNA Extraction, Library Preparation, and Sequencing

Following the manufacturer's protocol, using the QIAGEN DNeasy PowerSoil Pro Kit (Qiagen, Germantown, MD, USA), DNA from samples was isolated. Quantification of the extracted DNA samples was done using Qubit 4 fluorometer and Qubit™ dsDNA HS Assay Kit (Thermofisher Scientific, MA, USA).

Preparation of DNA libraries was done using the Nextera XT DNA Library Preparation Kit (Illumina, San Diego, CA, USA) and IDT Unique Dual Indexes with total DNA input of 1 ng. Using a proportional amount of Illumina Nextera XT fragmentation enzyme, genomic DNA was fragmented. To each sample, unique dual indexes were added and then libraries were constructed after 12 cycles of PCR. With AMpure magnetic Beads (Beckman Coulter, Brea, CA, USA) DNA libraries were purified and eluted in QIAGEN EB buffer. Libraries were then sequenced on an Illumina NovaSeq 6000 System with S4 Flow Cell.

### Bioinformatics Analysis

Unassembled sequencing reads were directly analyzed using CosmosID-HUB Microbiome Platform (CosmosID Inc., Germantown, MD, USA). In short, the platform employed curated genome databases together with a high-performance data-mining algorithm allowing rapid disambiguation of hundreds of millions of metagenomic sequence reads into the discrete microorganisms engendering the particular sequences.

Raw data was backed up to Amazon AWS and run through fastqc for quality checks upon data generation. A multiqc report, which was checked to ensure the conformation of read depth thresholds, and there was no abnormality with read quality, duplication rates, or adapter content, was generated. Taxonomic results were checked on the http://app.cosmosid.com platform to ensure no contamination nor barcoding issues existed. For statistical significance of the results, the filtering threshold was based on statistical scores determined by analyzation of a large number of diverse metagenomes.

Relative abundance of species of Bifidobacterium and Bacteroides was employed for our analyses. To avoid biases from outliers, the double median absolute deviation approach (Leys C, Ley C, Klein O, Bernard P, Licata L., J Exp Soc Psychol., 2013, 49 (4): 764-6) was employed to remove outliers. Subsequently, relative abundance of each species was summed over all the samples from all the subjects and only the species with a summation greater than one were used in our analyses since including species with infinitesimal abundance does not convey much information and could hamper the efficiency of the analysis with an unnecessarily larger number of variables.

### Mullen Scales of Early Learning (MSEL)

The MSEL, a validated and widely used infant cognitive development assessment tool, comprises of five subdomains: fine motor, gross motor, visual reception, receptive language, and expressive language (Mullen EM. Mullen scales of early learning. AGS Circle Pines, MN; 1995). An early learning composite score which is consistent with the Developmental Quotient score for infants is derived using all subdomain scores excluding gross motor. Trained staff administered the MSEL assessment at every visit.

### Statistical Modeling and Analyses

The R version 4.0.3 was used for all statistical analyses. Relative abundances of the selected species were used. Interaction terms were included to capture the dependence between infant gut microbiota and HMOs. The relative abundances and the HMO concentrations were first standardized to ensure fair comparisons among all gut microbiota species and HMOs prior to subsequent analyses. Specifically, the following models including no stratification and stratification based on A-tetra status in HM were fitted, respectively, and adjusting covariates included infant sex, delivery mode, maternal education, site, and batch of HMO analyses.

$$Unstratified$$

$$Mullen = {β}_{0} + {β}_{1} adjusting covariates + {β}_{2} HMO + {β}_{3} MB + {γ}_{1} HMO \times MB + ϵ$$

$$Stratified based on A - tetra +$$

$$Mullen = {β}_{0} + {β}_{1} adjusting covariates + {β}_{2} HMO ( Atetra + ) + {β}_{3} MB + {γ}_{1} HMO ( Atetra + ) \times MB + {β}_{4} I ( Atetra - ) + ϵ$$

$$Stratified based on A - tetra -$$

$$Mullen = {β}_{0} + {β}_{1} adjusting covariates + {β}_{2} HMO ( Atetra - ) + {β}_{3} MB + {γ}_{1} HMO ( Atetra - ) \times MB + {β}_{4} I ( Atetra + ) + ϵ$$

Here, HMO and MB correspond to the HMO quantifications and the relative abundances of the species from Bifidobacterium genera, respectively. Using the unstratified model, all HMO measures from all subjects were used. Thus, the estimated effects were associations between HMOs and infant cognition. In contrast, for the stratified models, HMO (Atetra+) and HMO(Atetra−) indicate HMO as a function of A-tetra status, where HMO (Atetra+) represents the HMO quantifications from the HM with detectable A-tetra (> the limit of detection of 4.4 mg/L), and vice versa. Furthermore, a binary indicator representing if the HM samples contained detectable (I(Atetra+)) or undetectable (I(Atetra−)) A-tetra was included for the stratified models and/(Atetra+)=1 when the HM samples were used for A-tetra+ subjects, and 0 for the A-tetra-subjects and vice versa for I(Atetra−). Using the stratified models, only HMOs from specific groups of subjects were used for the regression models. Thus, the estimated effects captured the associations between infant cognition and HMOs from homogeneous subjects depending on the A-tetra status. Collectively, if consistent significant associations between HMOs and cognition were observed for all three models, it implied that the associations were independent of A-tetra status or otherwise the associations depended on the A-tetra status.

Finally, to determine how HMOs and gut microbiota and their interactions may be associated with cognition, a two-step approach was employed, which included variable selection and regression analyses. Specifically, the group least absolute shrinkage and selection operator (LASSO) (Lim M, Hastie T., J Comput Graph Stat., 2015, 24 (3): 627-54.) was first used for variable selection of HMOs, microbitota species and their interactions for subsequent regression analyses. This step was needed to reduce dimensionality of the included variables and minimize overfitting. Due to the randomness of group LASSO, the selected variables could vary from each selection step. Thus, group LASSO was repeated 200 times and the variables used in the model with the smallest root mean squared error were chosen. If the final selected model did not include any interaction terms, the main effects with all HMOs and gut microbiota relative abundances were used for subsequent regression analyses. Finally, linear mixed effects models were fitted with the chosen variables to account for the longitudinal data (Laird N M, Ware J H., Biometrics., 1982, 38(4):963-74; and Verbeke G, Molenberghs G, Rizopoulos D. Random Effects Models for Longitudinal Data. In: van Montfort K, Oud J H L, Satorra A, editors. Longitudinal Research with Latent Variables [Internet]. Berlin, Heidelberg: Springer Berlin Heidelberg; 2010. p. 37-96. Available from: https://doi.org/10.1007/978-3-642-11760-2_2). The dependence within a subject was captured with a random intercept for each infant.

### Image Acquisition

All images were acquired using a 3T Siemens scanner. Specifically, T1-weighted images were acquired with the following parameters: TR=2400 ms, TE=2.24 ms, and resolution of 0.8×0.8×0.8 mm3. T2-weighted images were acquired using the following parameters: TR=3200 ms, TE=564 ms, and resolution of 0.8×0.8×0.8 mm3. Resting-state functional MRI (rs-fMRI) were acquired using the echo-planar imaging (EPI) sequence with the following parameters: TR=800 ms, TE=37 ms, resolution of 2×2×2 mm3, and 420 volumes.

### Image Preprocessing

For each subject, anatomical images were segmented into gray matter, white matter, and cerebrospinal fluid (CSF), using infant brain extraction and analysis toolbox (iBEAT) algorithm. Tissue segmentation images were further normalized to a standard template using the advanced normalization tools (ANTs).

Rs-fMRI scans were preprocessed using an infant-dedicated pipeline, including motion correction, distortion correction, bandpass filtering, and a deep-learning based independent component analysis (ICA)-denoising step. Subsequently, rs-fMRI data were linearly aligned to the corresponding T1 images first and then wrapped to the MNI space using the deformation field from ANTs algorithm.

### Network Construction

We constructed infant-dedicated network masks using the seed regions reported by Smith et al. (2009), including the medial visual network (V1), the occipital pole network (V2), the lateral visual network (V3), the default mode network (DMN), the sensorimotor network (SM), the auditory network (AN), the salience network (SA), and two lateralized frontoparietal networks (FPN-L, FPN-R) (Smith et al., PNAS, 2009, 106:13040-13045). Specifically, for each network seed, we constructed the network mask, based on 216 BCP subjects (Howell et al., Neurolmage, 2019, 185:891-905; Cho et al., Am. J. Clin. Nutr., 2021, 114:588-597; and Li et al., Front. Nutr., 2022, 9:919769) at 1 year old, by detecting significant connections from the seed to each voxel (p<0.05, FDR corrected). Then, for each subject, we calculated the averaged Pearson's correlation between each seed and all voxels within the mask, which we further named “network functional connectivity”.

### Example 1

A total of 105 infants between 3 and 12 months old (mean and standard deviation of age: 8.09±2.48 months) were included in this study. Of the 105 infants, 60 visited once while the remaining subjects had at most four visits, leading to a total of 170 MSEL assessments, HM samples, and fecal samples. The assessments and samples were analysed as described above.

Expressive language showed significant positive associations with the interaction between LNFP-I and B. kashiwanohense (p=0.048; ES=6.31) in the A-tetra-stratified model. This effect was not observed with either LNFP-I or B. kashiwanohense independently. Furthermore, this effect was not observed with the interaction between LNFP-I and any other Bifidobacterium species which were tested in this study (data not shown).

The A-tetra status of HM depends both on secretor positive status and blood type of A or AB leading to a subgrouping of secretor positive HM (Sabharwal H, Sjöblad S, Lundblad A., J Pediatr Gastroenterol Nutr., 1991, 12 (4); Lefebvre G, Shevlyakova M, Charpagne A, Marquis J, Vogel M, Kirsten T, et al., Front Nutr., 2020, 7, available from: https://www.frontiersin.org/articles/10.3389/fnut.2020.574459; and Austin S, De Castro C A, Benet T, Hou Y, Sun H, Thakkar S K, et al., Nutrients, 2016, 8 (6): 346). In our study, we found significant associations differed according to the stratification based on A-tetra detectability. When the associations of the microbiota are dependent on HMOs through interactions, the associations are more significant when HMOs are stratified by A-tetra detectability. This is intuitive in the sense that when stratified, the HMO components would be more homogenous, and thus would have less deviation, allowing more detection of significant terms.

Expressive language is the ability to communicate and express thoughts and feelings, while receptive language concerns understanding of information (Bloom L. Talking, understanding, and thinking: Developmental relationship between receptive and expressive language. 1974). The present inventors have previously reported that both receptive and expressive language abilities were significantly and positively associated with 3′-SL in infants received HM containing detectable A-tetra but not in A-tetra negative group (Cho S, Zhu Z, Li T, Baluyot K, Howell B R, Hazlett H C, et al., Am J Clin Nutr, 2021, 114 (2): 588-97). These previous findings, however, differed from the current studies. The potential interaction between HMOs and gut microbiota was not accounted for in our previous study. The identified associations with language ability in this study were through interactions between microbiota and HMOs. Contrasting to our previous findings, which were specific to A-tetra+group, inclusion of the interactions between HMOs and microbiota yielded previously unseen associations. That is, our findings underscore the importance of considering the potential interactions between HMOs and gut microbiota in the analyses of potential associations between HMOs and cognition.

### Example 2

In this study, a total of 91 subjects with 153 longitudinal scans were included in the analysis, age ranging between 0.3 and 20 months.

Neural connections, providing the substrate for functional networks, exist whether or not they are functionally active at any given moment (Smith et al., PNAS, 2009, 106:13040-13045). We constructed infant-dedicated network masks using the seed regions reported by Smith et al. (2009), including the medial visual network (V1), the occipital pole network (V2), the lateral visual network (V3), the default mode network (DMN), the sensorimotor network (SM), the auditory network (AN), the salience network (SA), and two lateralized frontoparietal networks (FPN-L, FPN-R). Specifically, for each network seed, we constructed the network mask, based on 216 BCP subjects at 1 year old, by detecting significant connections from the seed to each voxel (p<0.05, FDR corrected). Then, for each subject, we calculated the averaged Pearson's correlation between each seed and all voxels within the mask, which we further named “network functional connectivity”. These analyses are also described in more detail above.

The functional network strength can be evaluated as how strongly are the parts of the functional network interrelated.

Based on these seeds used in the study, we can hypothesize the following functions.

The sensorimotor network (SM) corresponds closely to the activations seen in bimanual motor tasks. This corresponds most strongly to the action-execution and perception-somesthesis paradigms. Thus, benefits associated with this domain are motor function, dexterity, motor tasks.

The auditory network (AN) corresponds most strongly to action-execution-speech, cognition-language-speech, and perception-audition paradigms. Thus, benefits associated with this domain are audition and language perception.

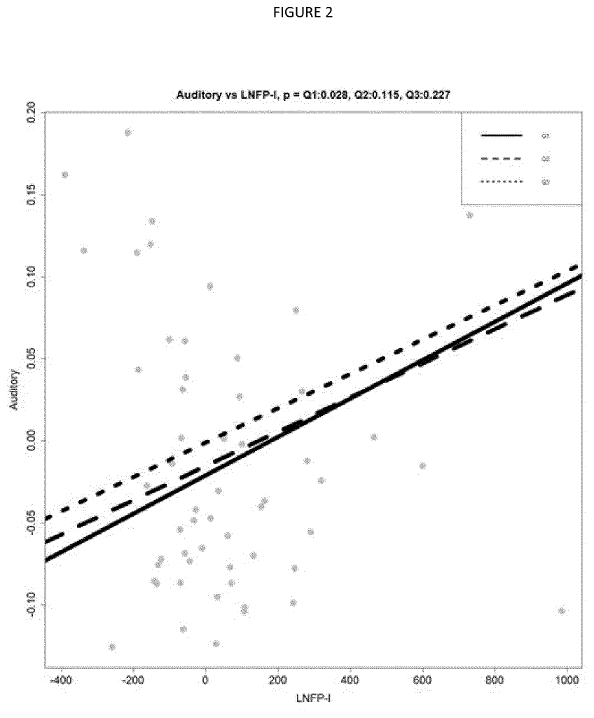

The results show a significant positive association between LNFP-I and sensorimotor (FIG. 1), auditory (FIG. 2), and default mode brain networks (data not shown), which can be linked to benefits in motor function, dexterity, motor tasks, audition and language perception.

### Example 3

The inventors investigated the association of motor skills and cognition in infants at 6 and 12 months with milk concentrations of specific human milk oligosaccharides (HMOs).

The following 24 individuals HMOs were collected in breastmilk at two- and three-months post-partum for a total cohort of 60 infants, analysed and quantified using ultra-high-performance liquid chromatography with fluorescence detection (UHPLC-FLD) according to the method of Austin S, Benet T. (Quantitative determination of non-lactose milk oligosaccharides. Anal Chim Acta. 2018 Jun. 20; 1010:86-96. doi: 10.1016/j.aca.2017.12.036. Epub 2017 Dec. 30. PMID: 29447675): 2′-Fucosyllactose (2′FL), 3′-Galactosyllactose (3′GL), 3′-Sialyllactose (3′SL), 3,2′-Difucosyllactose (DFL), 3-Fucosyllactose (3FL), 6′-Galactosyllactose (6′GL), 6′-Sialyllactose (6′SL), A-Tetrasaccharide (A-TETRA), Difucosyllacto-N-Hexaose-A (DFLNHa, Disialyllacto-N-Tetraose (DSLNT), Hex4 HexNAc2, Lacto-N-Difucohexaose-I (LNDFH-I), Lacto-N-Fucopentaose-I (NFP-I), Lacto-N-Fucopentaose-II (LNFP-II), Lacto-N-Fucopentaose-III (LNFP-III), Lacto-N-Fucopentaose-V (LNFP-V), Lacto-N-Hexaose (LNH), Lacto-N-Neodifucohexaose (LNnDFH), Lacto-N-Neofucopentaose-V (LNnFP-V), Lacto-N-Neotetraose (LNnT), Lacto-N-Tetraose (LNT), Monofucosyllacto-N-Hexaose-III (MFLNH-III), Sialyllacto-N-Tetraose-B (LSTb), Sialyllacto-N-Tetraose-C(STc). 2′FL, 3FL, 3′SL, 6′SL, LNT, LNnT, and LNFP-I were quantified against genuine standards of analytical quality, all other HMOs were quantified against maltotriose assuming equimolar response factors.

Infant motor skills and cognition were scored on the Bayley-III Scales (Bayley N., 2006. Bayley Scales of Infant and Toddler Development, Third Edition. San Antonio, TX: Psychorp) including sub-scores relating to fine and gross motor capacities at 6 months of age. A regression model approach was used where the covariates included: maternal net income, SES (social economic status)/education of mothers, weight, gestational age, sex of the infant (covariates were imputed, missing values are replaced by the population mean).

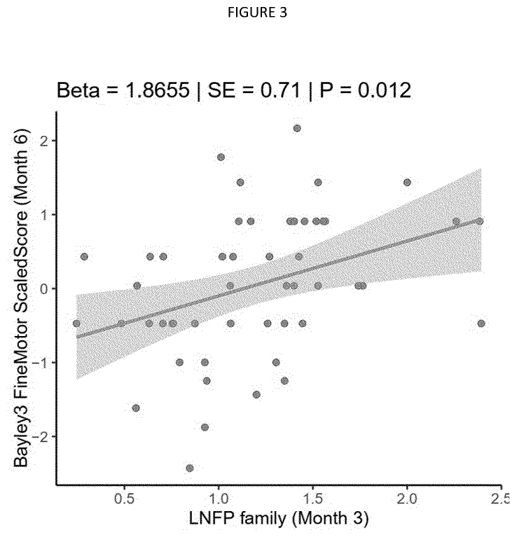

Out of those 24 HMOs, the combination of LNFP-I, LNFP-II and LNFP-III in the mother's breastmilk are associated with positive fine motor skills in infants at 6 months of age (see Table 1 below and FIG. 3).

TABLE 1

| | Bayley's | | | |
|---|---|---|---|---|
| HMOs | Motor Scores | Infant age | P | N |
| LNFP-I, II, III | Increased fine | 6 months | 0.012 | 50 |
| (Collected at 3 months) | motor scores | | | |

The inventor's findings surprisingly show an association of the specified combination of HMOs (LNFP-I, -II, -III) in the mother's breastmilk at two- and three-months post-partum with positive fine motor skills (i.e. improved fine motor skills) in infants at 6 months of age.

An improvement in motor skills is particularly beneficial in a subject aged 6 months, as this is a pivotal age for key milestones for motor skills (see e.g. Scharf R. J., Scharf G. J., Stroustrup A., 2016. Pediatr Rev. 37 (1), pp 25-37) which includes taking a second object while holding on to the first.

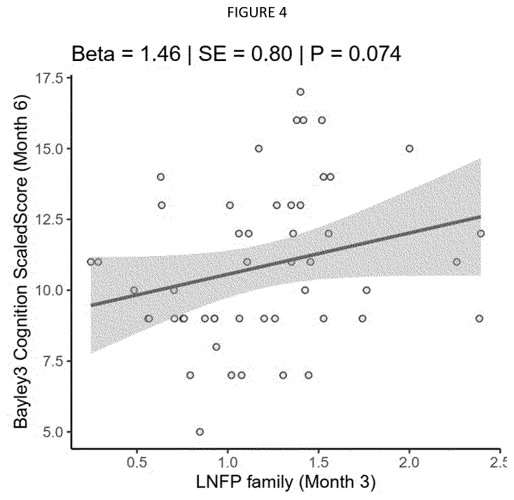

A similar trend was observed on cognition (FIG. 4): LNFP-I, -II, -III in mother's milk at two- and three-months post-partum is trending towards an association with positive cognition score on the Bayley-III scales at 6 months of age (p=0.074).

Thus, the inventor's findings surprisingly show a trend towards an association of the specified combination of HMOs (LNFP-I, -II, -III) in the mother's breastmilk with positive cognition (i.e. improved cognition) in infants at 6 months of age.

1: A method of improving brain development and/or cognitive development in a subject, the method comprising administering a nutritional composition comprising at least one lacto-N-fucopentaose (LNFP) to the subject.

2: The method according to claim 1, wherein the improvement in brain development and/or cognitive development comprises an improvement in development of auditory brain networks, sensorimotor brain networks, default mode brain networks, language, fine motor skills, or combinations thereof.

3: The method according to claim 2, wherein the improvement in the development of language comprises an improvement in receptive language and/or expressive language based on a score of Mullen sub-scale or of the Bayley sub-scale.

4: The method according to claim 2, wherein the improvement in the development of language comprises an improvement in expressive language based on a score of Mullen sub-scale or of the Bayley sub-scale.

5: The method according to claim 1, wherein the improvement in the brain development and/or the cognitive development comprises an improvement in expressive language based on a score of Mullen sub-scale or of the Bayley sub-scale.

6: The method according to claim 5, wherein the nutritional composition is administered in an amount effective for increasing an abundance of Bifidobacterium kashiwanohense in the gastrointestinal tract of the subject.

7: The method according to claim 6, wherein the subject is:
a) born of an A-tetrasaccharide negative mother; and/or
b) the subject is fed with a mother's milk lacking A-tetrasaccharide and/or the at least one LNFP or with a mother's milk having the at least one LFNP at a concentration of 100 mg/mL or less.

8: The method according to claim 1, wherein the at least one LNFP is present in a total amount from 25 mg/L to 5000 mg/L of the nutritional composition or from 0.02-3.75 g/100 g of the nutritional composition.

9: The method according to claim 1, wherein the nutritional composition further comprises B. kashiwanohense.

10: The method according to claim 1, wherein the subject is an infant, a young child or a child, and/or wherein the nutritional composition is an infant formula, a starter infant formula, a follow-on or follow-up infant formula, a baby food, an infant cereal composition, a growing-up milk, a fortifier or a supplement.

11. (canceled)

12: A method of increasing the abundance of Bifidobacterium kashiwanohense in the gastrointestinal tract of a subject, the method comprising administering a nutritional composition comprising at least one lacto-N-fucopentaose (LNFP) to the subject.

13. (canceled)

14: A method of improving brain development and/or cognitive development in a subject, the method comprising:
(i) obtaining mother's milk of the subject;
(ii) analysing the mother's milk for a presence of A-tetrasacharide and/or at least one lacto-N-fucopentaose (LNFP);
(iii) if the A-tetrasacharide and/or the at least one LNFP is absent or the at least one LNFP is present at a concentration of 100 mg/mL or less from the mother's milk, supplementing the mother's milk with a nutritional composition comprising the at least one LNFP; and
(iv) administering the supplemented milk to the subject,
wherein the subject is an infant, a young child or a child, and
wherein the improvement in brain development and/or cognitive development comprises an improvement in language development, or comprises an improvement fine motor skills.

15: A nutritional composition comprising at least one lacto-N-fucopentaose (LNFP) and Bifidobacterium kashiwanohense.

16: The method according to claim 1, wherein the at least one lacto-N-fucopentaose (LNFP) comprises lacto-N-fucopentaose-I (LNFP-I), lacto-N-fucopentaose-II (LNFP-II), lacto-N-fucopentaose-III (LNFP-III), lacto-N-fucopentaose-V (LNFP-V), or any combination thereof.

17: The method according to claim 1, wherein the at least one lacto-N-fucopentaose (LNFP) comprises lacto-N-fucopentaose-I (LNFP-I).

18: The method according to claim 1, wherein the at least one lacto-N-fucopentaose (LNFP) comprises or consists of lacto-N-fucopentaose-II (LNFP-II).

19: The method according to claim 1, wherein the at least one lacto-N-fucopentaose (LNFP) comprises lacto-N-fucopentaose-III (LNFP-III).

20: The method according to claim 1, wherein the at least one lacto-N-fucopentaose (LNFP) comprises or consists of lacto-N-fucopentaose-V (LNFP-V).

21: The method according to claim 1, wherein the at least one lacto-N-fucopentaose (LNFP) comprises a combination of lacto-N-fucopentaose-I (LNFP-I), lacto-N-fucopentaose-II (LNFP-II), lacto-N-fucopentaose-III (LNFP-III), and lacto-N-fucopentaose-V (LNFP-V).

22: The method according to claim 1, wherein the at least one lacto-N-fucopentaose (LNFP) comprises a combination of lacto-N-fucopentaose-I (LNFP-I), lacto-N-fucopentaose-II (LNFP-II) and lacto-N-fucopentaose-III (LNFP-III).

23: The method according to claim 1, wherein the at least one lacto-N-fucopentaose (LNFP) comprises a combination of lacto-N-fucopentaose-I (LNFP-I) and lacto-N-fucopentaose-II (LNFP-II).

24: The method according to claim 1, wherein the at least one lacto-N-fucopentaose (LNFP) comprises a combination of lacto-N-fucopentaose-I (LNFP-I) and lacto-N-fucopentaose-III (LNFP-III).

25: The method according to claim 1, wherein the at least one lacto-N-fucopentaose (LNFP) comprises a combination of lacto-N-fucopentaose-II (LNFP-II) and lacto-N-fucopentaose-III (LNFP-III).

26: The method according to claim 1, wherein the at least one lacto-N-fucopentaose (LNFP) comprises:
a combination of lacto-N-fucopentaose-I (LNFP-I), lacto-N-fucopentaose-II (LNFP-II) and lacto-N-fucopentaose-III (LNFP-III); or
lacto-N-fucopentaose-I (LNFP-I).
